(19) United States
(12) Patent Application Publication
(10) Pub. No.: US 2026/0240215 A1
(43) Pub. Date: Aug. 20, 2026

(54) BACILLUS VELEZENSIS STRAIN FOR IMPROVING HEALTH AND PERFORMANCE OF LIVESTOCK

(71) Applicant: KEMIN INDUSTRIES, INC., Des Moines, IA (US)

(72) Inventors: Michael BELVERUD, Pleasant Hill, IA (US); Mitchell POSS, Johnston, IA (US); Jon RUBACH, West Des Moines, IA (US); Sally MOORE, Ankeny, IA (US)

(73) Assignee: KEMIN INDUSTRIES, INC., Des Moines, IA (US)

(21) Appl. No.: 19/544,801

(22) Filed: Feb. 19, 2026

Related U.S. Application Data
(60) Provisional application No. 63/760,476, filed on Feb. 19, 2025.

(51) Int. Cl. A23L 33/135; A23K 10/18; A23K 20/174; A61K 35/74; A61K 35/742; A61K 35/747; C12N 1/20

(52) U.S. Cl. CPC A23L 33/135; A23K 10/18; A23K 20/174; A61K 35/74; A61K 35/742; A61K 35/747; C12N 1/20

(57) ABSTRACT

The present invention relates to a novel B. velezensis strain that improves the health and performance of animals. Another aspect of the present invention relates to using the novel strain to inhibit or reduce pathogen growth in animal feed, for instance as an animal feed additive. Another aspect of the present invention relates to a direct-fed microbial composition, wherein administration of the composition results in an improved performance parameter. Another aspect of the present invention relates to a novel strain that can be incorporated into food or foodstuff.

### CROSS-REFERENCE TO RELATED APPLICATIONS

This application claims priority to U.S. Provisional Application 63/760,476, BACILLUS VELEZENSIS STRAIN FOR IMPROVING HEALTH AND PERFORMANCE OF LIVESTOCK.

### REFERENCE TO A DEPOSIT OF BIOLOGICAL MATERIAL

This application contains a reference to a deposit of biological material, which deposit is incorporated by reference.

### FIELD OF THE INVENTION

The present invention relates to a novel Bacillus velezensis strain and its use as a direct-fed microbial.

### BACKGROUND OF THE INVENTION

Direct-fed microbials (DFMs), also called probiotics, are intended to improve the intestinal health of the animals that consume them. An ideal isolate should be able to withstand the high temperatures (80-90° C.) of pelleting, protect against pathogens, and improve performance metrics such as bodyweight gain and feed efficiency. Key criteria for any new isolate that may be commercialized are strong in vitro efficacy against numerous pathogens, and production of enzymes that can aid in digestion. The use of Bacillus strains as probiotic ingredients in the feed industry is well known in the art. The gram-positive spore-forming bacteria of the genus Bacillus have been favored because of their ability to survive pelleting at high temperatures (90° C.).

Enzymes produced and secreted by probiotics can be beneficial to livestock by hydrolyzing molecules that can be absorbed through the intestines. Amylase, xylanase, protease, and lipase break down starch, xylan, protein, and fats into glucose, xylose, amino acids, and free-fatty acids. These probiotic-produced enzymes can improve feed efficiency and growth performance.

There is therefore a need for probiotics which influence the gut microflora in a positive way and assist with the digestion of specific feed ingredients.

For these and other reasons, there is a need for the present invention.

### SUMMARY OF THE INVENTION

The present invention relates to the discovery that the addition of DFM from Bacillus velezensis species to animal feed can be used to prevent and/or control livestock pathogens, including E. Coli, Salmonella, Enterococcus, and other common livestock pathogens. The Bacillus velezensis species can also improve the body weight gain and/or feed efficiency. The Bacillus velezensis species may also decrease necrotic enteritis and mortality.

The present invention also relates to compositions comprising the Bacillus velezensis strain according to the invention which improves health and performance of production animals.

The invention further relates to the novel strain Bacillus velezensis having the deposit accession number PTA-127643; a strain having all of the identifying characteristics of Bacillus velezensis PTA-127643 or a mutant thereof. In some embodiments, the mutant has a DNA sequence with at least 95% similarity, preferably at least 98%, 99%, or 99.5% similarity.

In one aspect the invention relates to a Bacillus velezensis strain characterized in that:
i) the Bacillus velezensis strain has high levels of lipase, amylase, protease, and xylanase activity;
ii) the Bacillus velezensis strain has enzymatic activity under aerobic and/or anaerobic conditions that hydrolyzes one or more substrates selected from the group consisting of starch, xylan, protein, and fats into glucose, xylose, amino acids, and free-fatty acids;
iii) the Bacillus velezensis strain shows strong in vitro inhibition of common livestock pathogens; and
iv) the Bacillus velezensis strain improves feed efficiency and/or growth performance and/or other performance parameters in livestock and chickens fed with the Bacillus velezensis strain.

In a preferred embodiment the invention relates to a composition, e.g., comprising a carrier and a Bacillus velezensis strain wherein:
i) the Bacillus velezensis strain has high levels of lipase, amylase, protease, and xylanase activity;
ii) the Bacillus velezensis strain has enzymatic activity under aerobic and/or anaerobic conditions that hydrolyzes one or more substrates selected from the group consisting of starch, xylan, protein, and fats into glucose, xylose, amino acids, and free-fatty acids;
iii) the Bacillus velezensis strain shows strong in vitro inhibition of common livestock pathogens;
iv) the Bacillus velezensis strain improves feed efficiency and/or growth performance and/or other performance parameters in livestock and chickens fed with the Bacillus velezensis strain; and
v) the Bacillus velezensis strain having the deposit accession number PTA-127643; a strain having all of the identifying characteristics of Bacillus velezensis PTA-127643 or a mutant thereof.

In some embodiments, the composition is an animal feed or an animal feed additive. In some embodiments, the composition is a human food product.

### BRIEF DESCRIPTION OF THE DRAWINGS

FIG. 1 shows mean±SE lipopolysaccharide binding protein (ng/μL) in calves on days 0, 10, and 16 from Example 5.

FIG. 2 shows the feed intake results from Example 6.

FIG. 3 shows the adjusted FCR results from Example 6.

FIG. 4 shows the weight gain results from Example 6.

### DETAILED DESCRIPTION OF THE INVENTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise. It should be appreciated that aspects of the disclosure that are described with respect to a system are applicable to the methods, and vice versa, unless the context explicitly dictates otherwise.

Numeric ranges disclosed herein are inclusive of their endpoints. For example, a numeric range of between 1 and 10 includes the values 1 and 10. When a series of numeric ranges are disclosed for a given value, the present disclosure expressly contemplates ranges including all combinations of the upper and lower bounds of those ranges. For example, a numeric range of between 1 and 10 or between 2 and 9 is intended to include the numeric ranges of between 1 and 9 and between 2 and 10.

The present invention is directed to bacteria that are effective at inhibiting the growth of hemolytic E. coli and other pathogens in livestock.

Bacillus velezensis D3 (“D3”) is a novel probiotic microorganism. The strain was deposited on Aug. 16, 2023 under the terms of the Budapest Treaty on the International Recognition of the Deposit of Microorganisms for the Purpose of Patent Procedure. Deposit was made by Kemin Industries, Inc. at the ATCC Patent Depository 10801 University Boulevard, Manassas, Virginia 20110 USA, which assigned it Accession Number PTA-127643.

In a first embodiment, the current invention is directed to a Bacillus velezensis strain and preparations that contain or are derived from this strain or mutants thereof. Specifically, the invention is directed to one or more of the following groups:
a) the Bacillus velezensis strain deposited under PTA-127643 at the ATCC;
b) a mutant of the Bacillus velezensis strain deposited under PTA-127643 having all identifying characteristics of the strain PTA-127643, wherein the mutant preferably has a DNA sequence identity to PTA-127643 of at least 95%, preferably at least 96, 97 or 98%, and more preferably at least 99 or 99.5%;
c) a preparation containing (a) or (b);
d) a preparation containing an effective mixture of metabolites from (a), (b) or (c).

The strain of the current invention and mutants thereof is preferably characterized by at least one, and more preferably by all, of the following further features:
i) the Bacillus velezensis strain has high levels of lipase, amylase, protease, and xylanase activity;
ii) the Bacillus velezensis strain has enzymatic activity under aerobic and/or anaerobic conditions that hydrolyzes one or more substrates selected from the group consisting of starch, xylan, protein, and fats into glucose, xylose, amino acids, and free-fatty acids;
iii) the Bacillus velezensis strain shows strong in vitro inhibition of common livestock pathogens; and
iv) the Bacillus velensis strain reduces the need for antibiotics
v) the Bacillus velezensis strain improves feed efficiency and/or growth performance in livestock and chickens fed with the Bacillus velezensis strain.

The strain is preferably further characterized by a lipase activity of at least 190 U/L, more preferably at least 193 U/L, amylase activity of at least 40 U/L, more preferably at least 41 U/L, protease activity of at least 40 μg/mL, more preferably at least 44 μg/mL, and xylanase activity of at least 50 U/mL, more preferably at least 54 U/ml.

In addition, the strains of the current invention preferably survive the high temperatures necessary for pelleting animal feed, in particular, they preferably survive a temperature of at least 80° C. for at least 20 minutes.

Without wishing to be bound by any theory, it is thought that the Bacillus velezensis strain according to the current invention enhance animal health by a multifaceted mode of action, including the production and secretion of enzymes that can then break down starch, xylan, protein, and fats into glucose, xylose, amino acids, and free-fatty acids, thereby improving feed efficiency and growth performance. The Bacillus velezensis strain further competes with pathogenic bacteria by better consuming the available nutrients, thereby suppressing effective establishment of pathogenic bacteria in the gut.

It is an advantage of probiotics in comparison to antibiotics, that they do not destroy bacteria indiscriminately and do not they lead to antibiotic resistant strains of pathogenic bacteria. Normally, they are able to selectively compete with pathogenic bacteria by production of antimicrobial substances with specific efficacy, and are ideally able to simultaneously enhance the growth and viability of beneficial gut microflora. Further, they are preferably able to stimulate a systemic immune response in the treated animals.

The mutant strains of PTA-127643 of the current invention are preferably spontaneous mutants. The term “spontaneous mutant” refers to mutants that arise from PTA-127643 without the intentional use of mutagens. Such spontaneous mutants may be obtained by classical methods, such as growing the Bacillus velezensis strain in the presence of UV light or in the presence of a certain antibiotic to which the parent is susceptible and testing any resistant mutants for improved biological activity or improved ability to enhance one or more of the indicia of animal health. Other methods for identifying spontaneous mutants are known to those of ordinary skill in the art. But besides these preferred spontaneous mutants all other kinds of mutants of PTA-127643, e.g., mutants obtained by genetic engineering, are also part of the current invention.

One particular embodiment of the current invention are mutants of the strain PTA-127643 that are not found in nature and that have the characteristics described herein.

In a preferred embodiment of the current invention, the strains and preparations of the present invention are administered orally to animals, including livestock and production animals. In alternative embodiments, the strains and preparations of the present invention are administered orally to humans.

Thus, a further subject of the current invention are compositions, such as feedstuffs, foodstuffs, drinking and rearing water as well as therapeutic compositions, containing a B. velezensis strain and/or a preparation of the current invention.

A further subject of the current invention is also the use of a B. velezensis strain and/or a preparation of the current invention as a probiotic ingredient (DFM) in feed or food products.

Preferred foodstuffs according to the invention are dairy products, in particular yogurt, cheese, milk, butter and quark.

The cells of the strains of the current invention may be present, in particular in compositions as spores (which are dormant), as vegetative cells (which are growing), as transition state cells (which are transitioning from growth phase to sporulation phase) or as a combination of at least two, and preferably all of these types of cells. In a preferred embodiment, the composition of the current invention comprises mainly or only spores. In some embodiments, the invention comprises a biologically pure culture of B. velezensis. In some embodiments, the invention comprises an isolated B. velezensis strain.

The Bacillus velezensis strain of the current invention and compositions containing them, when administered to animals, preferably enhance the health of such animals and/or improve the general physical condition of such animals and/or improve the feed conversion rate of such animals and/or decrease the mortality rate of such animals and/or increase the survival rates of such animals and/or improve the weight gain of such animals and/or increase the productivity of such animals and/or increase the disease resistance of such animals and/or increase the immune response of such animals and/or establish or maintain a healthy gut microflora in such animals and/or reduce antibiotic use in such animals and/or reduce the pathogen shedding through the feces of such animals. In particular, the strains and compositions of the current invention might be used to assist in re-establishing a healthy balance of the gut microflora after administration of antibiotics for therapeutic purposes.

A further subject of the current invention is therefore a method of enhancing the health of animals and/or of improving the general physical condition of animals and/or of improving the feed conversion rate of animals and/or of decreasing the mortality rate of animals and/or of increasing the survival rates of animals and/or of improving the weight gain of animals and/or of increasing the productivity of animals and/or of increasing the disease resistance of animals and/or of increasing the immune response of animals and/or of establishing or maintaining a healthy gut microflora in animals and/or reduce antibiotic use in animals and/or of reducing the pathogen shedding through the feces of animals, wherein the strains and/or preparations of the current invention or the compositions of the current invention, which comprise such strain(s), are administered to animals.

A further subject of the current invention is therefore also the use of strains and/or preparations and/or compositions of the current invention for enhancing the health of animals and/or for improving the general physical condition of animals and/or for improving the feed conversion rate of animals and/or for decreasing the mortality rate of animals and/or for increasing the survival rates of animals and/or for improving the weight gain of animals and/or for increasing the productivity of animals and/or for increasing the disease resistance of animals and/or for increasing the immune response of animals and/or for establishing or maintaining a healthy gut microflora in animals and/or reduce antibiotic use in animals and/or for reducing the pathogen shedding through the feces of animals, wherein the strains and/or preparations of the current invention or the compositions of the current invention, which comprise such strain(s), are administered to animals.

A further subject of the current invention is therefore also the strains and preparations of the current invention as mentioned before and the compositions of the current invention, containing those strains, for enhancing the health of animals and/or for improving the general physical condition of animals and/or for improving the feed conversion rate of animals and/or for decreasing the mortality rate of animals and/or for increasing the survival rate of animals and/or for improving the weight gain of animals and/or for increasing the productivity of animals and/or for increasing the disease resistance of animals and/or for increasing the immune response of animals and/or for establishing or maintaining a healthy gut microflora in animals and/or reduce antibiotic use in animals and/or for reducing the pathogen shedding through the feces of animals.

“Increasing the productivity of animals” may refer, in particular, to any of the following: production of more or higher quality eggs, milk or meat or increased production of weaned offspring. Such increase may be measured by any standard metric or performance parameter, including but not limited to body weight gain, European production efficacy factor, feed conversion ratio, animal health or egg yield.

The methods and uses of the strains, preparations and compositions of the current invention can be therapeutic or non-therapeutic. In a particularly in at least one preferred embodiment of the current invention, the methods and uses are non-pharmaceutic, in particular, feeding applications.

Untreated manure of animals may have a detrimental environmental effect due to pathogenic bacteria and other ingredients, particularly with respect to the animals themselves and/or with respect to human beings coming in contact with the manure. This detrimental effect can be alleviated by either feeding the animals, or directly treating the manure or the bedding of the animals with the strains, compositions or preparations of the current invention. Therefore, a further subject of the current invention is a method of controlling and/or avoiding detrimental environmental effects of manure or contaminated liquids, the method comprising the step of applying to manure, contaminated liquids, litter, a pit, or a manure pond at least one strain, one preparation and/or one composition according to the current invention. Preferably, the composition is applied in liquid form, for example by spraying, or as a powder, for example by strewing.

Detrimental bacteria may have a negative influence on the consistency of litter and in particular may effect a rather fluid or highly fluid litter, which might lead to foot pad lesions of poultry and which can be avoided by feeding the animals with the strains, compositions or preparations of the current invention. Therefore a further subject of the current invention is a method of controlling and/or improving the consistency of litter, in particular a method of ensuring a solid consistency of litter and/or a method of avoiding foot pad lesions, the method comprising the step of feeding animals, in particular poultry, at least one strain, one preparation and/or one composition according to the current invention.

The strains and preparations according to the present invention can also be used for improving the quality of water. A further subject of the current invention is therefore also a method of controlling and/or improving the quality of water or aqueous solutions, in particular of drinking water and/or rearing water, comprising the step of applying to water or an aqueous solution at least one strain and/or at least one preparation and/or at least one composition of the current invention.

Further, the strains and preparations according to the invention can also be used for treating microbial diseases of plants. A further subject of the current invention is therefore also a method of treating and/or preventing microbial diseases of plants, in particular of cultivated plants, comprising the step of applying to the plants at least one strain and/or at least one preparation and/or at least one composition of the current invention. The application may be carried out in liquid form, such as by spraying, or in solid form, in particular as a powder.

By using the strains, preparations and compositions of the current invention preferably an improvement of at least one of the features mentioned above is realized, wherein realization of the feature preferably means an improvement of at least 1%, more preferably of at least 3 or at least 5%, in comparison to an adequate negative control. As a negative control, averages known in the animal husbandry field may be used, but preferably animals which are subjected to the same treatment as the animals tested are used, but without administration of the strains and/or preparations of the current invention.

In particular, the strains, preparations and compositions of the current invention may be administered or fed to an animal in an amount effective to inhibit and/or decrease the growth of pathogenic bacteria in the animal gut. Such pathogenic bacteria include hemolytic E. coli, S. Typhimurium, S. Litchfield, S. Enterica, S. Montevideo, S. Panama, S. Schwarzengrund, S. aureus, E. gallinarium, S. agalactiae, S. enteritidis, S. Senftenberg, R. equi, S. equi, S. Derby, E. cecorum, C. perfringens, C. difficile, C. septicum, S. suis, and other species of clostridia and salmonella. Similarly, the strains, preparations, and compositions of the current invention may be administered or fed to an animal in an amount sufficient to inhibit and/or decrease the prevalence of coccidiosis and/or necrotic enteritis. Relatedly, the methods of the present invention may be used to decrease the pathogenic bacteria shed in animal feces. The methods of the present invention may also be used to maintain or increase the growth of beneficial bacteria, such as lactic acid bacteria, in the animal gut. By decreasing pathogenic bacteria and/or increasing or maintaining beneficial bacteria, the compositions of the present invention are able to maintain an overall healthy gut microflora,
Thus, a further subject of the current invention is a method of inhibiting and/or decreasing the growth of harmful or pathogenic bacteria and/or maintaining and/or increasing the growth of beneficial bacteria in an animal gut, wherein strains and/or preparations and/or compositions of the current invention are administered to animals and wherein the pathogenic bacteria are preferably selected from hemolytic E. coli, S. Typhimurium, S. Litchfield, S. Enterica, S. Montevideo, S. Panama, S. Schwarzengrund, S. aureus, E. gallinarium, S. agalactiae, S. enteritidis, S. Senftenberg, R. equi, S. equi, S. Derby, and E. cecorum.

In at least one preferred embodiment of the invention, the compositions of the present invention reduce the amount of at least one pathogenic bacterium by at least 0.5 log, and more preferably by at least 1 log, 2 log, or 3 log. In an embodiment, the compositions of the present invention can reduce in vitro growth of pathogenic isolates by at least 75% in 12 hours, at least 80% in 12 hours, at least 85% in 12 hours, at least 90% in 12 hours, at least 95% in 12 hours, at least 75% in 20 hours, at least 80% in 20 hours, at least 85% in 20 hours, at least 90% in 20 hours, at least 95% in 20 hours.

Thus, a further subject of the current invention are the strains, preparations and compositions of the current invention for inhibiting and/or decreasing the growth of pathogenic bacteria and/or for maintaining and/or increasing the growth of beneficial bacteria in an animal gut, wherein the pathogenic bacteria are preferably selected from hemolytic E. coli, S. Typhimurium, S. Litchfield, S. Enterica, S. Montevideo, S. Panama, S. Schwarzengrund, S. aureus, E. gallinarium, S. agalactiae, S. enteritidis, S. Senftenberg, R. equi, S. equi, S. Derby, and E. cecorum.

Another aspect of the present invention relates to a therapeutic composition comprising the strains and/or compositions of the current invention as described herein. By way of non-limiting example, the present invention relates to a therapeutic composition for treatment and/or prevention of necrotic enteritis, in particular sub-clinical necrotic enteritis, in animals, preferably poultry, comprising the strains and/or compositions of the current invention as described herein.

Another aspect of the present invention relates to a therapeutic composition for treatment and/or prevention of bacterial enteritis, gangrenous dermatitis, colangiohepatitis, clostridiosis, diarrhea and/or foot pad dermatitis, in animals, preferably poultry, comprising the strains and/or compositions of the current invention as previously mentioned.

Another aspect of the present invention relates to a method of treating and/or preventing and/or ameliorating the symptoms associated with a disease, in particular of a gut disease, preferably of necrotic enteritis, in particular of sub-clinical necrotic enteritis, in poultry, wherein a strain and/or composition and/or preparation of present invention is administered to an animal in need thereof.

Another aspect of the present invention relates to a method of treating and/or preventing and/or ameliorating the symptoms associated with a disease, preferably a disease of poultry, selected from bacterial enteritis, gangrenous dermatitis, colangiohepatitis, diarrhea and/or foot pad dermatitis, wherein a strain and/or composition and/or preparation of the current invention is administered to an animal in need thereof.

In another embodiment, the Bacillus velezensis strain of the invention results in the production and secretion of enzymes that are beneficial to livestock by hydrolyzing molecules that can then be absorbed through the intestines. In one embodiment, the enzymes produced include one or more and not limited to amylase, xylanase, protease, and lipase that break down one or more and not limited to starch, xylan, protein, and fats into glucose, xylose, amino acids, and free-fatty acids. In at least one embodiment, the enzymes produced include lipase and amylase in particular.

In at least one embodiment, the Bacillus velezensis strain of the present invention results in the reduction of antibiotic use or administration to the animal. In some embodiments, the Bacillus velezensis strain of the invention decreases the need for antibiotics by limiting the ability of hemolytic bacteria to grow. The reduction in antibiotic use may be compared to untreated animals or animals treated with another composition.

The strains and/or preparations and/or compositions of the present invention can be administered to animals in feed and/or drinking water over multiple days throughout the animal's life or during particular stages or portions of the animal's life. For example, the strains and/or compositions can be administered only in a starter diet or only in a finisher diet of livestock or production animals.

In another aspect of the invention, the administration of the Bacillus velezensis strain PTA-127643 as described herein has been shown to provide numerous benefits to animal health and performance. Specifically, the methods of the invention are effective for improving body weight gain and feed conversion ratio. Furthermore, the administration of the strain can improve the overall health of the animal, which includes, but is not limited to, reducing mortality. The methods are also effective for the prophylactic prevention of diseases such as necrotic enteritis. In animals already experiencing a pathogenic infection, the administration of the strain can reduce the severity of associated symptoms. A significant commercial and health advantage is that the administration of the strain can decrease the overall need for therapeutic antibiotic use in an animal production system by improving the animal's baseline health and resistance to infection.

Another aspect of the present invention relates to a method of enhancing the health of human beings and/or of improving the general physical condition of human beings and/or of increasing the disease resistance of human beings and/or of increasing the immune response of human beings and/or of establishing or maintaining a healthy gut microflora in human beings, wherein the strains and/or preparations of the current invention or the compositions of the current invention are administered to human beings.

Another aspect of the present invention relates to use of strains and/or preparations and/or compositions of the current invention for enhancing the health of human beings and/or for improving the general physical condition of human beings and/or for increasing the disease resistance of human beings and/or for increasing the immune response of human beings and/or for establishing or maintaining a healthy gut microflora in human beings, wherein the strains and/or preparations of the current invention or the compositions of the current invention, which comprise such strain(s), are administered to human beings.

The compositions of the present invention, in particular the feed, food and pharmaceutical compositions as well as the drinking or rearing water, preferably comprise the strains of the current invention and are administered to animals at a rate of about 1×10² to about 2×10¹² CFU/g feed or mL water, in particular in a rate of about 1×10³ or about 1×10⁴ or about 1×10⁵ or about 1×10⁶ or about 1×10⁷ or about 1×10⁸ or about 1×10⁹ or about 1×10¹⁰ or about 1×10¹¹ or about 1×10¹² CFU/g feed or mL water, preferably in an amount of about 1×10³ and about 1×10¹⁴ CFU/g feed or mL water or about 1×10⁴ to about 1×10¹⁰ CFU/g feed or ml water, and more preferably in an amount of 1×10⁴ to 1×10⁷ CFU/g feed or ml water.

The compositions of the present invention, in in particular the feed, food and pharmaceutical compositions as well as the drinking or rearing water, preferably comprise the strains of the current invention and are administered to animals at a rate of about 0.5 to 2.0 g/head/day. More preferably at a rate of about 1 g/head/day.

Correspondingly, preferred amounts of the strains and/or preparations of the current invention in the feed, food and water compositions of the current invention range preferably from 0.1 wt.-% to 10 wt.-%, more preferably from 0.2 wt.-% to 5 wt.-%, in particular from 0.3 wt.-% to 3 wt.-%.

The methods of the present invention may be used for all kind of animals, in particular all kind of non-human and non-insect animals, more preferably all kind of vertebrates such as mammals, aquatic animals and birds.

Animals that may benefit from the current invention include but are not limited to farm animals, pets, exotic animals, zoo animals, aquatic animals, animals used for sports, recreation or work. Pets are preferably selected from dogs, cats, domestic birds and domestic exotic animals.

Aquatic animals are preferably selected from finfish and crustaceans which are preferably intended for human nutrition. These include, in particular, carp, tilapia, catfish, tuna, salmon, trout, barramundi, bream, perch, cod, shrimps, lobster, crabs, prawns and crayfish. Preferred types of salmon in this context are the Atlantic salmon, red salmon, masu salmon, king salmon, keta salmon, coho salmon, Danube salmon, Pacific salmon and pink salmon.

Further preferred aquatic animals are fanning fish which are subsequently processed to give fish meal or fish oil. In this connection, the fish are preferably herring, pollack, menhaden, anchovies, capelin or cod.

In a further preferred embodiment, the animals are farm animals (livestock or production), which may be raised for consumption as food or dairy, such as poultry, swine, ruminants and equine (horses). The poultry may be selected from productive or domestic poultry, but also from fancy poultry or wild fowl. Preferred productive poultry in this context are chickens, turkeys, ducks and geese. The productive livestock in this context is preferably poultry optimized for producing young stock or poultry optimized for bearing meat. Preferred fancy poultry or wild fowl are peacocks, pheasants, partridges, chukkars, guinea fowl, quails, capercaillies, grouse, pigeons and swans, with quails being especially preferred. Further preferred poultry are ratites, in particular ostriches and emus, as well as parrots.

Ruminants according to the current invention are preferably selected from bovine (cattle), goat, sheep, and deer. In one embodiment, the compositions of this invention may be fed to pre-ruminants to enhance their health and, in particular, to decrease the incidence of diarrhea in these animals pre-ruminants are ruminants, including calves, ranging in age from birth to about twelve weeks.

The compositions of the current invention may comprise at least one carrier or typical feed ingredients or combinations thereof. Suitable carriers are inert formulation ingredients added to improve recovery, efficacy, or physical properties and/or to aid in packaging and administration. Such carriers may be added individually or in combination. These carriers may be selected from anti-caking agents, flowability agents, anti-oxidation agents, bulking agents, surfactants, emulsifiers, binders, and/or protectants. Examples of useful carriers include polysaccharides (in particular starches, maltodextrins, methylcelluloses, gums, chitosan and/or inulin's), protein sources (in particular skim-milk powder and/or sweet-whey powder), peptides, sugars (in particular lactose, trehalose, sucrose and/or dextrose), lipids (in particular lecithin, vegetable oils and/or mineral oils), salts (in particular sodium chloride, sodium carbonate, calcium carbonate, chalk, limestone, magnesium carbonate, sodium phosphate, calcium phosphate, magnesium phosphate and/or sodium citrate), and silicates (in particular clays, in particular beolite clay, amorphous silica, fumed/precipitated silicas, zeolites, Fuller's earth, baylith, clintpolite, montmorillonite, diatomaceous earth, talc, bentonites, and/or silicate salts like aluminum, magnesium and/or calcium silicate). Suitable carriers for animal feed additives are set forth in the American Feed Control Officials, Inc.'s Official Publication, which publishes annually See, for example Official Publication of American Feed Control Officials, Sharon Krebs, editor, 2006 edition, ISBN 1-878341-18-9. These carriers may include dextrin, silicon, bentonite, leonhardite, and zeolite. The carriers can be added after concentrating the fermentation broth and/or during and/or after drying. Preferred carriers according to the invention are selected from calcium carbonate, diatomaceous earth and vegetable oil.

A preferred embodiment of the current invention are concentrate compositions, in particular feed additive compositions, i.e. compositions suitable for preparing a feed composition, which comprise at least one strain of the current invention and at least one carrier, wherein the at least one strain is preferably comprised in an amount of 0.1 to 10 wt.-%, more preferably in an amount of 0.2 to 5 wt.-%, in particular in an amount of 0.3 to 3 wt.-%, and most preferably in an amount of 0.4 to 2.2 wt.-%, and the at least one carrier is preferably comprised of an amount of at least 90 wt. %, preferably in an amount of 90 to 99.9 wt.-%, more preferably in an amount of 95 to 99.8 wt.-%, in particular in an amount of 97 to 99.7 wt.-%, and most preferably in an amount of 97.8 to 99.6 wt.-%, and wherein the carrier consists preferably substantially of limestone, in particular of limestone with smaller parts of diatomaceous earth and/or vegetable oil.

These preferred compositions of the current invention, which contain stabilized strains, can be used for the preparation of feed and pharmaceutical compositions as well as drinking and rearing water which preferably comprise the strains according to the invention in an amount as mentioned above. In a preferred embodiment, 200 to 1000 grams of such a concentrate composition, in particular 250, 500 or 1000 grams of such a concentrate composition, are used per ton of feed, drinking or rearing water to provide compositions which can be used for feeding animals These concentrate compositions preferably comprise at least one strain of the current invention in an amount of 1×10⁹ to 2×10¹¹ CFU, in particular 2×10⁹ to 1×10¹¹ CFU, per g of the concentrate composition. Starting from these concentrate compositions, feed and food compositions can be prepared by mixing the concentrate compositions with typical feed or food ingredients, respectively.

Typical animal feed ingredients which may be contained in the compositions according to the invention and/or used in the preparation of feed compositions starting from concentrate compositions according to the invention include one or more of the following: proteins, carbohydrates, fats, further probiotics or microbes, prebiotics, enzymes, vitamins, immune modulators, milk replacers, minerals, amino acids, coccidiostats, acid-based products and/or medicines, such as antibiotics.

The compositions of the invention, such as an animal feed or animal feed additive, may further comprise an agriculturally acceptable carrier. The carrier may be selected from one or more of water, glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, maltodextrin, white oil, corn cob meal, rice bran, glucose, sucrose, sorbitol, lactose, wheat flour, wheat bran, corn gluten meal, starch, and cellulose. The composition may also comprise at least one additional enzyme, prebiotic, or probiotic. In some embodiments, the composition is provided as a supplement or milk replacer.

Carbohydrates containing components which may be used according to the invention are for example forage, roughage, wheat meal, sunflower meal or soya meal, and mixtures thereof.

Protein-containing components which may be used according to the invention are for example soya protein, pea protein, wheat gluten or corn gluten, and mixtures thereof.

Fat-containing components which may be used according to the invention are in particular oils, of both animal and plant origin, like vegetable oils, for example soya bean oil, rapeseed oil, sunflower seed oil, flaxseed oil or palm oil, fish oil, and mixtures thereof.

Protein-containing components which additionally contain fats which may be used according to the invention are for example fish meal, krill meal, bivalve meal, squid meal or shrimp shells, as well as combinations thereof.

Further probiotics (DFM) which may be used according to the invention in combination with the strains and preparations of the invention are preferably other strains of bacteria selected from the species Bacillus velezensis, Bacillus licheniformis, Bacillus lentus, Bacillus pumilus, Bacillus laterosporus, Bacillus coagulans, Bacillus alevi, Bacillus cereus, Bacillus badius, Bacillus thurigiensis, Enterococcus faecium, and Pediococcus acidilactici. Other preferred bacteria are Bacillus velezensis PB6 (as described in U.S. Pat. No. 7,247,299 and deposited as ATCC Accession No. PTA-6737), which is sold by Kemin under the trademark CLOSTAT®, Bacillus velezensis N23, deposited under accession number PTA-127793 by Kemin Industries, Bacillus velezensis C-3102 (as described in U.S. Pat. No. 4,919,936 and deposited as FERM BP-1096 with the Fermentation Research Institute, Agency of Industrial Science and Technology, in Japan), sold by Calpis as CALSPORIN®, Bacillus velezensis DSM 17299, as sold by Chr. Hansen under the trademark GalliPro®, a mixture of Bacillus velezensis DSM 17299 and Bacillus licheniformis DSM 17236, as sold by Chr. Hansen under the trademark GalliProTect®, a mixture of Bacillus licheniformis and Bacillus velezensis spores sold by Chr. Hansen under the trademark BIOPLUS2B®, Bacillus coagulans strains as described in U.S. Pat. No. 6,849,256, or Bacillus velezensis strain N23 sold by Kemin. Other non-Bacillus probiotics, such as Saccharomyces cerevisiae, Pichia pastoris, Aspergillus niger, Aspergillus oryzae, or Hansenula, may also be used in compositions of the present invention. In particular in food compositions further probiotics which are known to be useful to the human health may be used such as lactic acid producing bacteria, in particular lactobacilli, or Bifidobacteria. If said further probiotics are not formulated as part of the compositions of the present invention, they may be administered together (either at the same time or at different times) with the compositions of the present invention.

Prebiotics which may be used according to the invention are preferably oligosaccharides, in particular selected from galactooligosaccharides, silayloligosaccharides, lactulose, lactosucrose, fructooligosaccharides, palatinose or isomaltose oligosaccharides, glycosyl sucrose, maltooligosaccharides, mannanoligosaccharides, isomaltooligosaccharides, cyclodextrins, gentiooligosaccharides, soybean oligosaccharides, xylooligosaccharides, dextrans, pectins, polygalacturonan, rhamnogalacturonan, mannan, hemicellulose, arabinogalactan, arabinan, arabinoxylan, resistant starch, mehbiose, chitosan, agarose, inulin, tagatose, polydextrose, and alginate.

Enzymes which may be used in feed compositions according to the invention and which may further aid in the digestion of feed, are preferably selected from phytases (EC 3.1.3.8 or 3.1.3.26), xylanases (EC 3.2.1.8), galactanases (EC 3.2.1.89), galactosidases, in particular alpha-galactosidases (EC 3.2.1.22), proteases (EC 3.4), phospholipases, in particular phospholipases Al (EC 3.1.1.32), A2 (EC 3.1.1.4), C (EC 3.1.4.3), and D (EC 3.1.4.4), lysophospholipases (EC 3.1.1.5), amylases, in particular alpha-amylases (EC 3.2.1.1); lysozymes (EC 3.2.1.17), glucanases, in particular beta-glucanases (EC 3.2.1.4 or EC 3.2.1.6), glucoamylases, cellulases, pectinases, or any mixture thereof.

Examples of commercially available phytases include Bio-Feed™ Phytase (Novozymes), Ronozyme® P and HiPhos™ (DSM Nutritional Products), Natuphos™ (BASF), Finase® and Quantum® Blue (AB Enzymes), the Phyzyme® XP (Verenium/DuPont) and Axtra® PHY (DuPont). Other preferred phytases include those described in e.g. WO 98/28408, WO 00/43503, and WO 03/066847.

Examples of commercially available xylanases include Ronozyme® WX and G2 (DSM Nutritional Products), Econase® XT and Barley (AB Vista), Xylathin® (Verenium) and Axtra® XB (Xylanase/beta-glucanase, DuPont). Examples of commercially available proteases include Ronozyme® ProAct (DSM Nutritional Products).

Vitamins which may be used according to the invention are for example vitamin A, vitamin D3, vitamin E, vitamin K, e.g., vitamin K3, vitamin B12, biotin, choline, vitamin B1, vitamin B2, vitamin B6, niacin, folic acid and panthothenate, e.g., Ca-D-panthothenate, or combinations thereof.

Immune modulators which may be used are for example antibodies, cytokines, spray-dried plasma, interleukins, or interferons, or combinations thereof.

Minerals which may be used according to the invention are for example boron, cobalt, chloride, chromium, copper, fluoride, iodine, iron, manganese, molybdenum, selenium, zinc, calcium, magnesium, potassium, or sodium, or combinations thereof.

Amino acids which may be used according to the invention are for example lysine, alanine, threonine, methionine or tryptophan, or combinations thereof.

Natural oils/extracts which may be used in compositions according to the invention and which may further aid in the utility of the invention may include, but are not limited to, clove oil, oregano oil, monarda oil, spearmint extract and/or rosemary extract. If included, the natural oil/extract may comprise from about 0.05% to about 0.2% by weight of the composition, with about 0.10% to about 0.15% by weight being preferred. In one embodiment of the invention, the composition includes from about 0.05% to about 0.15% by weight clove oil.

Thus, a further embodiment of the current invention is a method of preparing an animal feed composition comprising mixing at least one strain and/or at least one preparation and/or at least one concentrate composition of the current invention, in an amount effective to enhance animal health, with feed ingredients, such as proteins, lipids and/or carbohydrates, and optionally further beneficial substances, as mentioned above, to provide a feeding product. This method may comprise for example also a pelleting step.

Standard pelleting processes known to those of skill in the art may be used, including extrusion processing of dry or semi-moist feeds. Preferred pelleting temperatures are between about 65° C. and about 120° C. The composition of the current invention can withstand the pelleting process while losing less than 1 log.

In an embodiment, the compositions of the current invention may be sprayed onto animals or onto animal feed (including, but not limited to via micro-machine), mixed into feed at a feed mill, or mixed into the water supply. In an embodiment, a powdered form of the bacterial strain is added from a microbin into a stream of water, which is blended into a homogenous liquid mixture with other dry products. The liquid mixture is then applied to the feed by directly pumping it onto the feed in the feed mixer.

The strains and compositions of the present invention can be obtained by culturing the strains of the current invention according to methods well known in the art, including by using the media and other methods as described for example in U.S. Pat. No. 6,060,051, EP0287699 or US2014/0010792. Conventional large-scale microbial culture processes include submerged fermentation, solid state fermentation, or liquid surface culture. Towards the end of fermentation, as nutrients are depleted, the cells of the strains begin the transition from growth phase to sporulation phase, such that the final product of fermentation is largely spores, metabolites and residual fermentation medium. Sporulation is part of the natural life cycle of these strains and is generally initiated by the cell in response to nutrient limitation. Fermentation is configured to obtain high levels of colony forming units of the Bacillus velezensis cells and to promote sporulation. The bacterial cells, spores and metabolites in culture media resulting from fermentation may be used directly or concentrated by conventional industrial methods, such as centrifugation, tangential-flow filtration, depth filtration, and evaporation. The concentrated fermentation broth may be washed, for example via a diafiltration process, to remove residual fermentation broth and metabolites.

The fermentation broth or broth concentrate can be dried with or without the addition of carriers using conventional drying processes or methods such as spray drying, freeze drying, tray drying, fluidized-bed drying, drum drying, or evaporation. The resulting dry products may be further processed, such as by milling or granulation, to achieve a specific particle size or physical format. Carriers, as described above, may also be added post-drying.

Preparations of the strains of the current invention may be cell-free preparations or preparations containing cell debris or preparations containing a mixture of intact cells and cell debris. Cell-free preparations of the strains of the current invention can be obtained for example by centrifugation and/or filtration of fermentation broth. Depending on the technique used, these cell-free preparations may not be completely devoid of cells, but may still comprise a smaller amount of cells. As the cells secrete compounds like metabolites, enzymes and/or peptides into the surrounding medium, the supernatant of the cells comprises a mixture of such compounds, in particular metabolites, enzymes and/or peptides, as secreted by the cells. Thus, in a preferred embodiment of the invention, the preparation of the strains is a supernatant of the fermentation broth.

Compositions comprising cell debris of the strains may be obtained by rupturing the cells applying techniques as known to those of skill in the art, for example by mechanical means or by applying high pressure. Depending on the degree of force applied, a composition comprising only ruptured cells or a composition comprising a mixture of cell debris and intact cells is obtained. Homogenization of the cells may be realized for example by utilizing a French cell press, sonicator, homogenizer, microfluidizer, ball mill, rod mill, pebble mill, bead mill, high pressure grinding roll, vertical shaft impactor, industrial blender, high shear mixer, paddle mixer, and/or polytron homogenizer. Suitable alternatives are enzymatic and/or chemical treatment of the cells.

Cell-free preparations of the current invention may comprise preparations which are obtained by first rupturing the cells by applying techniques as mentioned before and subsequently removing the cell debris and the remaining intact cells. Removing of the cell debris and remaining intact cells can be carried out in particular by centrifugation and/or filtration.

The preparations of the strains of the current invention may comprise active compounds such as at least one metabolite, preferably a mixture of metabolites, as further described below, and/or at least one enzyme selected from proteases, xylanases and/or cellulases, and/or at least one peptide, and/or combinations thereof.

A preparation containing an effective mixture of metabolites can be obtained for example according to the methods set forth in U.S. Pat. No. 6,060,051. In particular, the preparation can be obtained by precipitating the metabolites in the preparations mentioned using organic solvents like ethyl acetate and subsequently redissolving of the precipitated metabolites in an appropriate solvent. The metabolites may subsequently be purified by size exclusion filtration that groups metabolites into different fractions based on molecular weight cut-off.

Preferably an effective amount of the strains and/or preparations and/or compositions of the current invention is used in the embodiments of the current invention. The term “effective amount” refers to an amount which affects at least one beneficial effect to an animal and/or to the environment, in comparison to an animal or environment that has not been administered the strains and/or preparations and/or compositions of the current invention, but that has otherwise been treated the same.

In case of therapeutic applications, preferably a therapeutic amount of the strains and/or preparations and/or compositions of the current invention is used. The term “therapeutic amount” refers to an amount sufficient to ameliorate, reverse or prevent a disease state in an animal. Optimal dosage levels for various animals can easily be determined by those skilled in the art, by evaluating, among other things, the composition's ability to (i) inhibit or reduce pathogenic bacteria in the gut at various doses, (ii) increase or maintain levels of beneficial bacteria and/or (iii) enhance animal health at various doses.

The invention will now be illustrated by the following non-limiting Examples.

### EXAMPLES

### Example 1: Enzymatic Activity of Bacillus velezensis D3

### Materials and Methods

Preparation of cell-free supernatant. One gram of Bacillus velezensis PB6, Bacillus velezensis FXA, Bacillus lichiniformis G3, Bacillus velezensis D3, and ENTEROSURE® (a mix of lichiniformis and Bacillus velezensis) spores was transferred to individual 50 mL Falcon tubes. Saline (9 mL of 0.85%) was added to each tube and vortexed at high speed for three minutes. Each probiotic (1 mL) was transferred to 14 mL round bottom tubes containing saline (9 mL) to create a 100-fold dilution. Tubes were transferred to an 80° C. water bath for 10 minutes to kill vegetative cells. Tubes were subsequently transferred to chilled water to quickly cool the tubes. Each probiotic (1 mL) was transferred to individual flasks containing sterile Tryptic Soy Broth (TSB, Becton Dickinson, Sparks, MD) with 0.6% Yeast Extract (TSBYE, 100 mL). Flasks were incubated (New Brunswick Scientific Shaking Incubator, Model C24KC, Edison, NJ) overnight (~20-24 h) at 37° C. while shaking at 180 RPM under aerobic conditions. For N23 and C23, freezer stock (1 mL) was transferred to sterile TSBYE (100 mL) and incubated as stated above. Cultures (30 mL) of each overnight culture were transferred to 50 mL Falcon tubes and centrifuged with an Eppendorf 5804 R benchtop centrifuge (Eppendorf, Hamburg, Germany) for 10 minutes at 5000 RPM. Supernatant was decanted into 9 mL round bottom tubes and serially filtered with 0.45 and 0.22 μM syringe filters to make cell free supernatant, or CFS, which was then assayed for enzyme activity.

Xylanase activity. Xylanase activity was assayed according to SOP LB-IV-20/094-E². Reagents are listed in Table 1.

TABLE 1
Xylanase activity assay reagents.

| Reagent | Formula | Manufacturer | Part No. | Lot |
|---|---|---|---|---|
| Sodium Phosphate | Na₂HPO₄•2H₂O | Fisher | S472- | 178955 |
| Dibasic Dihydrate | | | 500 | |
| Potassium Phospahte | KH₂PO₄ | Fisher | P285-3 | 33426 |
| Monbasic | | | | |
| Citric Acid | C₆H₈O₇•H₂O | Fisher | A940- | 214888 |
| | | | 500 | |
| Beechwood Xylan | β-1,4,α-1,2-xylan | Sigma | X4252- | BCBH1776V |
| | | | 25G | |
| Sodium Carbonate | Na₂CO₃ | Fisher | S263-1 | 171716 |
| Anhydrous | | | | |
| Potassium Sodium | C₄H₄KNaO₆•4H₂O | Sigma | S2377- | 070M0108V |
| Tartrate Tetrahydrate | | | 1KG | |
| Sodium Bicarbonate | NaHCO₃ | Fisher | S233- | 170369 |
| | | | 500 | |
| Sodium Sulfate | Na₂SO₄ | Fisher | S421-1 | 195740 |
| Anhydrous | | | | |
| Copper (II) Sulfate | CuSO₄•5H₂O | Acros | 42361- | A008503201 |
| Pentahydrate | | | 5000 | |
| Sulfuric Acid, 95% | H₂SO₄ | Fisher | A300- | 168345 |
| | | | 212 | |
| Ammonium | (NH₄)₆Mo₇O₂₄•4H₂O | Fisher | A674- | 175 |
| Molybdate | | | 500 | |
| Tetrahydrate | | | | |
| Disodium Hydrogen | Na2HASO₄•7H₂O | Alfa Aesar | A18725 | Y22F034 |
| Arsenate | | | | |
| Xylose | C₅H₁₀O₅ | Sigma | X-1500 | 61H0760 |

Standard stock solution (2000 μg/mL) was prepared by dissolving 0.2±0.01 g of xylose in 100 mL of deionized (DI) water. To prepare standard solutions, the stock solution was diluted 10-fold to make a 200 μg/mL solution. This solution was further diluted according to Table 2.

TABLE 2
Xylanase standard preparation scheme.

| μg xylose/ | Diluted solution 200 | DI water |
|---|---|---|
| mL | μg/mL | (mL) |
| 0 | — | 1 |
| 40 | 0.2 mL | 0.8 |
| 80 | 0.4 mL | 0.6 |
| 120 | 0.6 mL | 0.4 |
| 160 | 0.8 mL | 0.2 |

CFS (1 mL) was dissolved in 9 mL of 0.1M phosphate buffer pH 7.5 and then extracted by mixing for 15 minutes on a magnetic stirrer. Solutions were centrifuged at 4600 RPM for 5 minutes to obtain a clear enzyme solution. Extracted CFS solutions (1 mL) were transferred to 15 mL Falcon tubes in duplicate. Blank tubes were left empty. Xylan substrate (0.5%, 1 mL) was added to each tube at 15-second intervals, vortexed for 15 seconds, and then placed in a 50° C. water bath. Tubes were incubated for 15 minutes at 50° C. Next, Nelson's reagent C (1 mL) was added to each tube (including blanks) and vortexed for 15 seconds to stop the reaction. Extracted CFS was added to blank tubes and vortexed for another 15 seconds. Tubes, including blanks, were placed in boiling water for 20 minutes. Tubes were then cooled, and Nelson's color reagent (1 mL) was added to all tubes, and then tubes were briefly vortexed. DI water (5 mL) was added to each tube and vortexed for 5 minutes. Samples were centrifuged at 3000 RPM to obtain a clear solution. Samples (1 mL) were transferred to cuvettes and read at 540 nm on a QuickDrop Spectrophotometer (Molecular Devices, Holliston, MA).

Amylase activity. Amylase activity was determined with Phadebas starch tablets (PN: 1301 Lot 3P5012). CFS (200 μL) was transferred to 14 mL round bottom tubes in duplicate. DI water (200 μL) was added to blank tubes. DI water (4 mL) was added to each tube. Tubes were placed in a 37° C. water bath for 5 minutes. Using forceps, one Phadebas tablet was added to each tube, which were then vortexed and placed back in the water bath for 15 minutes. Sodium hydroxide (0.5 M, 1 mL) was added to each tube, and tubes were vortexed and centrifuged at 1300×g for 5 minutes. Supernatant was transferred to cuvettes and read at 620 nm with a QuickDrop Spectrophotometer. Optical density (OD) values of the blanks were subtracted from the samples. Amylase activity (U/L) was determined using the standard curve included with the Phadebas tablets.

Protease activity. Protease activity was determined with a Pierce Protease Assay Kit (PN: 23263 Lot YL384777). Assay Buffer was prepared by dissolving the BupH Borate Buffer Pack in DI water (500 mL). Succinylated casein (1 vial) was dissolved in Assay Buffer (5 mL). Trypsin Stock Solution (50 mg/mL) was prepared by dissolving lyophilized TPCK Trypsin in DI water (1 mL). Trypsin Stock Solution was made into 50 μL aliquots and stored at −80° C. Trypsin Stock Solution was diluted 10-fold to 0.5 mg/mL and then diluted 2-fold to yield six standards. TNBSA (2,4,6-trinitrobenzene sulfonic acid) was prepared by adding TNBSA stock solution (100 μL) to 14.9 mL of Assay Buffer. In a 96-well clear-bottom microtiter plate, Succinylated Casein Solution (100 μL) was added to one set of wells. Assay Buffer (100 μL) was added to a duplicate set of wells to serve as blanks. Each CFS sample or standard (50 μL) was added to both the Succinylated Casein wells and the corresponding blank wells. The plate was incubated for 20 minutes at 37° C. TNBSA Working Solution (50 μL) was added to each well, and the plate was incubated for another 20 minutes at 37° C. Absorbance was measured at 450 nm with a Spectramax i3X microtiter plate reader (Molecular Devices, Sunnyvale, CA). Net absorbance was determined by subtracting the A450 of the blank from that of the corresponding Casein well. This ΔA450 value is the absorbance generated by the proteolytic activity of the CFS. Absorbance was plotted as ΔA450 against protease standard concentration to assess relative protease activity of the CFS.

Lipase Activity. Lipase activity was measured with an Abnova Lipase Assay Kit (PN: KA1654 Lot CEO1A11). The kit is based on an improved dimercaptopropanol tributyrate (BALB) method, in which SH groups formed from lipase cleavage of BALB react with 5,5′-dithio-bis(2-nitrobenzoic acid) (DTNB) to form a yellow colored product. The color intensity, measured at 412 nm, is proportionate to the enzyme activity in the sample. Working Reagent was prepared by combining Color Reagent (100 mg), Assay Buffer (280 μL), and BABL Reagent (160 μL). Calibrator and DI water (150 μL) were added to separate designated wells of a 96-well plate. CFS (10 μL) was added to appropriate wells followed by Working Reagent (140 μL). The plate was tapped to mix and read at 412 nm with a Spectramax i3X microtiter plate reader at 10 and 20 minutes. Lipase activity was determined by the below equation:

$$Activity = ( ( {OD}_{20 min} - {OD}_{10 min} ) / ( {OD}_{calibratior} - {OD}_{water} ) ) \times 735$$

where OD_{20 min} and OD_{10 min} are the OD_{412 nm} values of the sample at 20 and 10 minutes, respectively, and OD_{Calibrator} and OD_{water} are the OD_{412 nm} values of the calibrator and water at 20 minutes. The number “735” is the equivalent activity (U/L) of the calibrator under the assay conditions.

### Results

Lipase activity was highest in the PB6 CFS. Amylase and xylanase activity were highest in the CFS of N23, and protease activity was highest in the CFS from C23. Bacillus licheniformis (G3) CFS showed the lowest enzyme activity for all four enzymes. Complete results are in Table 3.

TABLE 3
Lipase, amylase, protease, and xylanase enzyme
activity in cell-free supernatant of D3, PB6, FXA, G3, C23,
N23 and ENTEROSURE ®. Mean ± Standard error (n = 3).

| | Lipase | Amylase | Protease | Xylanase |
|---|---|---|---|---|
| Probiotic | (U/L) | (U/L) | (ug/mL) | (U/mL) |
| D3 | 193 ± 0.01 | 41 ± 0.00 | 44 ± 0.00 | 54 ± 0.00 |
| (Bacillus | | | | |
| velezensis) | | | | |
| PB6 | 483 ± 0.02 | 247 ± 0.02 | 39 ± 0.00 | 36 ± 0.00 |
| (Bacillus | | | | |
| velezensis) | | | | |
| FXA | 240 ± 0.00 | 199 ± 0.00 | 41 ± 0.00 | 46 ± 0.00 |
| (Bacillus | | | | |
| velezensis) | | | | |
| G3 | 74 ± 0.01 | 0.0 ± 0.00 | 29 ± 0.01 | 16 ± 0.01 |
| (Bacillus | | | | |
| licheniformis) | | | | |
| ENT | 306 ± 0.01 | 128 ± 0.00 | 37 ± 0.01 | 20 ± 0.01 |
| (Bacillus | | | | |
| velezensis | | | | |
| and Bacillus | | | | |
| licheniformis) | | | | |
| C23-2 | 396 ± 0.02 | 632 ± 0.00 | 47 ± 0.00 | 86 ± 0.00 |
| (Bacillus | | | | |
| velezensis) | | | | |
| N23-2 | 151 ± 0.00 | 716 ± 0.00 | 46 ± 0.00 | 108 ± 0.01 |
| (Bacillus | | | | |
| velezensis) | | | | |

### Discussion

Enzymes produced and secreted by probiotics can be beneficial to livestock by hydrolyzing molecules that can then be absorbed through the intestines. Amylase, xylanase, protease, and lipase break down starch, xylan, protein, and fats into glucose, xylose, amino acids, and free-fatty acids. These probiotic-produced enzymes can improve feed efficiency and growth performance. Swiatkiewicz, S. et al. (2014). Feed enzymes, probiotic, or chitosan can improve the nutritional efficacy of broiler chicken diets containing a high level of distillers dried grains with solubles. Livestock Science 163 (2014) 110-119.

PB6 CFS showed the most lipase activity of the probiotics tested, but N23 and C23 were the top producers of amylase, protease, and xylanase. Bacillus velezensis D3 has shown strong in vitro inhibition and was the third-highest producer of protease and xylanse, behind only C23 and N23. It was observed that D3 in vitro efficacy and secretion of lipase, amylase, protease, and xylanase make it a well-rounded probiotic with substantial promise.

### Example 2: In Vitro Inhibition from Bacillus velezensis D3

Table 4 shows the kinetic read microtiter inhibition results from D3 cell-free supernatant.

TABLE 4
Kinetic read microtiter percent inhibition: none (—),
1-25 (+), 26-50 (++), 51-75 (+++), and 76-100 (++++).

| | | D3 | D3 |
|---|---|---|---|
| Isolate | ID | 12 h | 20 h |
| Hemolytic E. coli | 2022030295 | ++++ | ++ |
| Hemolytic E. coli | 2021056941 | ++++ | ++++ |
| Hemolytic E. coli | 2021036616 | ++++ | ++++ |
| Hemolytic E. coli | 2023048026 | ++++ | ++++ |
| Hemolytic E. coli | 2023033094 | ++++ | ++++ |
| Hemolytic E. coli | 2023009091 | ++++ | ++++ |
| Hemolytic E. coli | 2023031314 | ++ + | ++ ++ |
| Hemolytic E. coli | 2023026033 | ++++ | ++++ |
| Hemolytic E. coli | 2023051522 | ++++ | ++++ |
| Hemolytic E. coli | 2023051522 | ++++ | ++++ |
| Hemolytic E. coli | 2021037480 | ++++ | ++++ |
| Hemolytic E. coli | 2023070969 | ++++ | ++++ |
| S. Typhimurium | M20-08867 | ++++ | ++++ |
| S. Litchfield | M21-05061-1 | ++++ | +++ |
| S. Enterica | M20-24110-1-4 | +++ | ++ |
| S. Montevideo | M19-34817 | ++++ | +++ |
| S. Enterica | M20-25381-1-7 | ++++ | +++ |
| S. Panama | M21-05061-2 | ++++ | +++ |
| S. Schwarzengrund | M21-28310 | ++++ | ++ |
| S. aureus | ATCC 29213 | ++ | + |
| S. aureus | ATCC 25923 | + | — |
| P. aeruginosa | ATCC 10145 | — | — |
| E. gallinarium | L23257007-6 | + | — |
| S. agalactiae | ATCC 12403 | ++++ | +++ |
| S. Montevideo | ATCC 8387 | + | ++ + |
| S. Typhimurium | ATCC 14028 | +++ + | ++++ |
| E. Coli | VDL-ISU 07-1690 | ++++ | ++++ |
| R. equi | VDL-ISU 45 | ++++ | ++++ |
| S. equi | VDL-ISU 43006 | ++++ | ++++ |
| S. Derby | ISU-SAL 0392-22 | ++++ | ++++ |
| E. cecorum | 21-87 | ++++ | ++++ |
| E. cecorum | 21-510 | ++++ | ++++ |
| E. cecorum | 21-1684 | ++++ | ++++ |
| E. cecorum | 21-1685 | ++++ | ++++ |

As demonstrated, D3 showed strong in vitro inhibition of select common livestock pathogens. Of the 37 pathogens tested, D3 reduced growth of 32 (86%) and 24 (65%) isolates by 75-100% at 12 and 20 h, respectively.

### Example 3: Evaluation of Bacillus velezensis D3 in a Natural E. coli Challenge Model in Piglets

Bacillus velezensis D3 was isolated from livestock tank water in May of 2012. In vitro testing with D3 has shown strong inhibition of several intestinal pathogens including E coli. E coli can cause colibacillosis or E. coli diarrhea, and septicemia, which can lead to morbidity and mortality, especially in young pigs. Blue River Research Services, LLC, Carthage IN, was selected to run a trial to evaluate the effect of supplementation of D3 in pig feed. The primary objective of this study was to evaluate the effect of D3 on reducing the impact of post weaning diarrhea as measured by fecal scores, E. coli quantification, and growth performance. The study was a randomized block design conducted in a multi-purpose house containing 80 total pens and consisted of five treatments in contiguous pens. Mecadox (50 g/ton), an antibiotic, was included as a positive control. There were three phases. Phase 1 was from day 0-7, phase 2, from day 7-21, phase 3, from day 21 to 42, and cumulative (d0-42).

Upon arrival, animals were assigned a unique identification, weighed, and randomized to treatment pens. Animals were weighed and rank ordered by body weight (high to low) within each gender. The heaviest 5 animals, within gender, were randomly assigned to the five pens within the first gender block, the second heaviest within gender were randomly assigned to the same block, and so on, until there were five animals of the same gender per pen. There were an equal number of male and female pens. Treatments were randomized within each gender block until each block contained each treatment. Animals were group housed throughout the study. The sow farm that the piglets were sourced from were confirmed positive for E. coli. Piglets were assigned to treatments when they were received at the study site (weaned at 21 d). To provide additional enteric stress, pens and feeders were rinsed. No disinfection was applied as recommended by BRRS.

Significant differences (p<0.05) were seen in average overall mortality. No differences were seen in fecal scores, growth performance or E coli prevalence from swab samples obtained on D1 and D19. Overall, Mecadox (50 g/ton) and D3 at E5, E6, and E7 CFU/g were ineffective at preventing an E coli outbreak, however, pigs fed D3 did have lower mortality than pigs fed Mecadox.

### Materials and Methods

Experimental treatments. D3 lot FEP-D3-2310001 was placed on calcium carbonate fre-flo (RM01029 Lot 2407108576) or provided as a concentrate. Two of the three treatments were prepared on calcium carbonate, one at 2.38E8 and one at 2.55E9 colony forming units (CFU) per gram. The third treatment was D3 concentrate and had a concentration of 1.95E10 CFU/g. A total of 3.5 kg of each treatment was sent for the trial. Each experimental treatment was assayed. In brief, 1:9 dilutions were prepared in 0.85% sterile saline, vortexed and further diluted 1:9. This dilution was heated to 80° C. for 10 min and immediately cooled in chilled water for two minutes. Further dilutions were made to the endpoint, plated in duplicate on 3M Aerobic Petrifilms (St. Paul, MN) and incubated overnight at 37° C. Mecadox (50 g/ton) was supplied by BRRS. Treatment details are shown in Table 5.

TABLE 5
Feed treatments and inclusion levels.

| Treatment | Treatment | Duration | No. of | Animals | Total # |
|---|---|---|---|---|---|
| No. | Description | (days) | Pens | per pen | Animals |
| 1 | Negative Control | 42 | 16 | 5 | 80 |
| 2 | Mecadox @ 50 g/ton | 42 | 16 | 5 | 80 |
| 3 | D3 @ 1E5 CFU/g | 42 | 16 | 5 | 80 |
| 4 | D3 @ 1E6 CFU/g | 42 | 16 | 5 | 80 |
| 5 | D3 @ 1E7 CFU/g | 42 | 16 | 5 | 80 |
| Total | | | 80 | | 400 |

Study design. The trial was conducted at Blue River Research Services, LLC in Carthage, IN Upon arrival, animals were uniquely identified, weighed, and randomized to treatment pens. Animals were weighed and rank ordered by body weight (high to low) within each gender. The heaviest five animals, within gender, were randomly assigned to the five pens within the first gender block, the second heaviest within gender randomly assigned to the same block, and so on, until there were five animals of the same gender per pen. There were an equal number of male and female pens.

Treatments were randomized within each gender block until each block contains each treatment. Animals were group housed throughout the study. Animal sourcing details were recorded in the study records. The sow farm that the piglets were sourced from were confirmed positive for E. coli. Piglets were assigned to treatments when they were received at the study site (weaned at 21 d). To provide additional enteric stress, pens and feeders were only rinsed. No disinfection was applied as recommended by BRRS.

A total of 400 Sus scrofa domesticus piglets were enrolled in the study, with five pigs in each of the 80 pens. Age was approximately three weeks, and weight was approximately 12-16 pounds. Males were castrated. Animals had approximately 4.8 ft²/animal of usable floor space. Each pen was 4′×6′. Animals received approximately 18 hours of light per 24-hour period. Temperature and fan settings were adjusted to meet recommended thermal conditions for each stage of growth per facility procedures. Ventilation was mechanical. Feed and water access was ad libitum. Health observations requiring an animal to be classified as “removed” were abnormalities that the observer considered: 1) resulting in pain or distress to the animal (e.g., a broken appendage), or 2) abnormalities that are likely to result in further deterioration of the animal's or its contemporaries' health (e.g., severe lameness), and/or 3) abnormalities that may impair the animal's ability to access food or water at the time of the observation (e.g., death, morbidity, or non-ambulatory).

“Removed” animals (e.g., culled and dead animals) were removed from the pen at the time of the observation or as soon as practical and a removal weight was obtained. When necessary, animals were euthanized. Necropsies were performed for all euthanized or dead animals, if deemed necessary. Animals were euthanized by designated personnel according to site specific procedures.

Feed preparation and Feed sample analysis. All basal and treatment feeds were manufactured at BRRS. For each feeding phase, a master batch was mixed, and all 5 treatment diets were derived. For each treatment batch, the respective test article was included in a premix (~50 lb.), which was then be incorporated in the middle of the treatment batch. Each treatment batch was mixed for ~10 minutes prior to discharging into 50 lb. labeled feed bags.

Three feed samples were collected: one from the beginning, middle, and end of the batch which was mixed to form two composite samples (primary and backup). One composite sample of each treatment diet was sent to Kemin Animal Nutrition and Health (KANA) research and development for analysis. Each bag was identified with the study number, date of bagging, and the treatment number. Feed samples were sent to Kemin for confirmation of the inclusion level of D3 before feeding commenced. Counting was done using the method stated in experimental treatments. All feed was fed in mash form.

Variables measured. Fecal scores, E. coli quantification/prevalence, lipopolysaccharide binding protein (LBP) levels in blood serum, fecal microbiome composition, average daily feed intake, average daily gain, and feed to gain.

Body weights. Weights per pen were performed on days 0, 7, 21 and 42.

Feed weigh-back. Remaining feed was weighed on days 7, 21 and 42.

Fecal scoring. Animals were observed daily throughout the study for clinical signs of diarrhea which was scored using a 5-point fecal scoring system.
1 None (normal feces)
2 Minimal (slightly soft feces)
3 Mild (soft, partially formed feces)
4 Moderate (loose, semi-liquid feces)
5 Severe (watery, mucous-like feces)

Scores for individual pens were recorded daily in the morning by a trained technician. Pens were assigned an overall fecal score based on the average score for that pen. Pigs with severe diarrhea were individually treated according to prescription of the attending veterinarian. All treatments were recorded, which will include treatment product and volume administered.

Fecal sample collection. Animals exhibiting clinical signs (fecal scores) of E. coli increases based on a considerable jump in the average fecal scores of pens (i.e. fecal score of 1 to changing to a fecal score of 3), had a composite fecal sample collected for each pen at 3-5 days post-break, from 2-3 pigs/pen. All samples were sent to Iowa State University Veterinary Diagnostic Laboratory (ISU VDL) veterinary diagnostic lab to be analyzed to detect relevant bacterial pathogens related to post-weaning diarrhoea. Bacteriological cultures were performed on fecal samples to detect E. coli. Positive E. coli samples were further analysed by PCR-genotyping to detect haemolytic strains of E. coli. This included testing for toxin genes: EAST1, STa, STb, Stx1, Stx2, Stx2e; pilus genes: F18, F41, K88, K99, 987P; and adhesion genes: AIDA, EAEA, PAA. All samples were labeled with study number, pen number, and date of collection. The rectal fecal sample from each pig was collected with new disposable gloves. Handlers did not use any lubricants if manual stimulation was needed. Approximately 1 to 3 grams of fresh feces was collected into a clean conical tubed and stored at −80° C. until ready to be shipped on dry ice for analysis.

Microbiome sample collection. Five composite fecal samples were collected from all treatments on the same day (day 20 or 21) of the above sample collection for E. coli testing. Care was taken to get the samples as fresh as possible. Composite samples were taken within 15 minutes of defecation. A minimum of 10 g of material was collected from the center of the dropping, which had not contacted the floor or air. Tubes were filled to minimize headspace. Samples were immediately placed on dry ice, then transferred to −80° C. Samples were stored at −80° C. until being shipped to Kemin on dry ice.

Upon receipt, samples were stored at −80° C. until being processed. Feces (~250 mg) was purified using a Qiagen Power Soil® Pro Kit (Ref.: 4706 Lot 175043714, Hilden Germany). Samples were purified per instructions in Kemin's Microbiome Sampling Protocol. Samples were eluted in Cytiva HyPure™ Molecular Biology Grade nuclease free water (80 μL, Hyclone Cat. No. SH30538.02 Lot AK30774141). Purified dsDNA (5 μL) was combined with Qubit™ 1×dsDNA BR Working Solution (195 μL, Ref.: Q33260 Lot 2725319) and briefly vortexed. Samples were quantified with a Qubit 4 Fluorometer (Invitrogen PN: Q33226 SN: 23226240100050). DNA purity was assessed by determining the 260/280 and 260/230 ratio using a Quick Drop (Molecular Devices PN: 504178, SN: 810039, Holliston, MA). DNA concentration was adjusted to 20 ng/μL before shipment to KAE for sequencing with Oxford Nanopore Technologies. Additionally, samples (8 μL) from each group were combined for metagenomic sequencing. All sequencing was performed by PathoSense, Ghent University, Ghent Belgium.

Lipopolysaccharide binding protein (LBP). BRRS obtained blood samples from three pigs per pen (80 pens) on days 8 and 22 of the study. Blood samples were centrifuged to separate blood cells from the serum, then the serum was stored at −80° C. until being shipped for analysis. An enzyme-linked immunosorbent assay was utilized (ELISA) kits (Abbexa PN: ABX154969, lot E2410154E, Cambridge, UK) specific for pig LBP, for quantification. To analyze, samples were allowed to warm to room temperature, diluted 100-fold in sodium and calcium free phosphate buffered saline (PBS, Dulbecco's sterile filtered, ATCC 30-2200, Lot 80419231). Samples were processed according to kit instruction then analyzed by measuring absorbance at 450 nm using an I3X spectrophotometer, Molecular Devices, Sunnydale, CA.

Performance and mortality parameters. Fecal samples were sent to ISU diagnostic laboratory for confirmation of E. coli challenge. Performance variables were average daily gain (ADG), average daily feed intake (ADFI), feed to gain (F/G) and mortality. Performance and mortality data were calculated for each of the three phases.

Statistical analysis. Data were evaluated using JMP (version 18.0.0), by analysis of variance (ANOVA) with comparison of means using Tukey t-test (p<0.05). Data were parsed using the following criteria. Means comparison bounds were set using a Huber K-sigma value of 4. Next, pens with two or less pigs remaining at the end of phase 2 were removed. Pens removed in phase 2 had data removed from phase 3 and cumulative results. Data from 13 pens were removed. Mortality data used results from all 80 pens.

### Results

D3 counts from each treatment in each phase were in-line with expected counts. See Table 6.

TABLE 6
D3 feed counts.

| | | | Actual |
|---|---|---|---|
| | | Target | Counts |
| Treatment | Phase | CFU/g | (CFU/g) |
| 1 and 2 | 1 | N/A | N/A |
| 3 | | 1E5 | 1.27E5 |
| 4 | | 1E6 | 9.83E5 |
| 5 | | 1E7 | 1.0E7 |
| 1 and 2 | 2 | N/A | N/A |
| 3 | | 1E5 | 9.85E4 |
| 4 | | 1E6 | 1.02E6 |
| 5 | | 1E7 | 1.03E7 |
| 1 and 2 | 3 | N/A | N/A |
| 3 | | 1E5 | 6.73E4 |
| 4 | | 1E6 | 6.62E5 |
| 5 | | 1E7 | 6.10E6 |

Overall (d0-42) mortality was high. The mortality rate in treatment 2 was 43.75% followed by treatment group 5, 4, 1 and 3 at 28.75, 22.5, 22.5, and 20.0 percent (Table 7).

TABLE 7

| Average percent mortality of pigs for days 0-42. Values not connected by | | |
|---|---|---|
| the same letter^{a-b} are significantly different (p < 0.05). | | |
| | | Percent |
| No. | Treatment | Mortality |
| 1 | Negative Control | 22.50^{b} |
| 2 | Mecadox @ 50 g/ton | 43.75ª |
| 3 | D3 @ 1E5 CFU/g | 20.00^{b} |
| 4 | D3 @ 1E6 CFU/g | 22.50^{b} |
| 5 | D3 @ 1E7 CFU/g | 28.75^{ab} |

E. coli prevalence testing of six composite fecal swabs collected on day 1 and sent to Iowa State University diagnostic lab for analysis and all were negative for E. coli. Further E. coli prevalence testing of 80 composite fecal samples collected on day 19 and sent to ISU were all positive except swabs from pigs in pens 25, 60, 69, and 78. Positive samples were all found to be hemolytic E. coli by genomic testing. Identified genes were EAST 1 (toxin), LT (toxin), STb (toxin), STx2 (toxin), Stx2e (toxin), F18 (pilus) and PAA 9 (adhesion). Not all samples were positive for all genes (Appendix 2).

There were two incidences during the trial were fecal scores increased significantly, with the first outbreak observed from Sep. 8, 2024 to Sep. 17, 2024, lasting nine days. The second incidence was from Sep. 18, 2024 to Oct. 3, 2024 which lasted 16 days. Average fecal scores for the two outbreaks are summarized in Table 8.

TABLE 8
Average fecal scores

| Treatment | Treatment | Sep. 8, 2024- | Sep. 18, 2024- |
|---|---|---|---|
| No. | Description | Sep. 17, 2024 | Oct. 3, 2024 |
| 1 | Negative Control | 1.5 | 2.4 |
| 2 | Mecadox @ 50 g/ton | 1.3 | 2.3 |
| 3 | D3 @ 1E5 CFU/g | 1.4 | 2.2 |
| 4 | D3 @ 1E6 CFU/g | 1.4 | 2.4 |
| 5 | D3 @ 1E7 CFU/g | 1.4 | 2.2 |

Average daily feed intake did not differ (p<0.05) for any phase. Results are shown in Table 9.

TABLE 9
Average Daily Feed Intake

| Treatment | | Phase | | | |
|---|---|---|---|---|---|
| No. | Treatment | 1 | 2 | 3 | Cumulative |
| 1 | Negative Control | 0.106 | 0.327 | 0.731 | 0.459 |
| 2 | Mecadox @ 50 | 0.114 | 0.368 | 0.817 | 0.498 |
| | g/ton | | | | |
| 3 | D3 @ 1E5 CFU/g | 0.099 | 0.335 | 0.711 | 0.458 |
| 4 | D3 @ 1E6 CFU/g | 0.118 | 0.342 | 0.734 | 0.476 |
| 5 | D3 @ 1E7 CFU/g | 0.109 | 0.323 | 0.723 | 0.448 |

Average daily gain did not differ (p<0.05) for any phase. Results are shown in Table 10.

TABLE 10
Average daily gain.

| Treatment | | Phase | | | |
|---|---|---|---|---|---|
| No. | Treatment | 1 | 2 | 3 | Cumulative |
| 1 | Negative Control | 0.048 | 0.189 | 0.387 | 0.259 |
| 2 | Mecadox @ 50 | 0.064 | 0.201 | 0.431 | 0.264 |
| | g/ton | | | | |
| 3 | D3 @ 1E5 CFU/g | 0.039 | 0.187 | 0.370 | 0.246 |
| 4 | D3 @ 1E6 CFU/g | 0.065 | 0.176 | 0.414 | 0.267 |
| 5 | D3 @ 1E7 CFU/g | 0.047 | 0.194 | 0.356 | 0.241 |

Feed efficiency, measured as feed to gain, did not differ (p<0.05) for any phase. Results are shown in Table 11.

TABLE 11
Feed to gain.

| Treatment | | Phase | | | |
|---|---|---|---|---|---|
| No. | Treatment | 1 | 2 | 3 | Cumulative |
| 1 | Negative Control | 2.226 | 1.793 | 1.744 | 1.826 |
| 2 | Mecadox @ 50 | 2.299 | 1.939 | 2.008 | 1.938 |
| | g/ton | | | | |
| 3 | D3 @ 1E5 CFU/g | 2.147 | 1.909 | 2.281 | 2.056 |
| 4 | D3 @ 1E6 CFU/g | 2.298 | 2.036 | 1.927 | 2.040 |
| 5 | D3 @ 1E7 CFU/g | 2.209 | 1.791 | 2.197 | 1.996 |

Serum LBP levels at both days (8 and 22) were highest in the negative control and were higher (p<0.05) than all treatment groups. Results are in Table 12.

TABLE 12
Analysis of Variance and connecting letter reports for serum LBP levels
on days 8 and 22. Values not connected by the same letter are
significantly different (p < 0.05).

| | | Day 8 LBP | Day 22 LBP |
|---|---|---|---|
| Treatment No. | Treatment | (ng/mL) | (ng/mL) |
| 1 | Negative Control | 895.0^{a} | 3991.8 |
| 2 | Mecadox @ 50 | 301.7^{b} | 293.9 |
| | g/ton | | |
| 3 | D3 @ 1E5 CFU/g | 304.7^{b} | 295.6 |
| 4 | D3 @ 1E6 CFU/g | 296.1^{b} | 293.4 |
| 5 | D3 @ 1E7 CFU/g | 372.1^{b} | 338.5 |
| | Standard Error | 115.1 | 355.6 |

### Discussion

Bacillus velezensis D3 has shown superior in vitro efficacy against Salmonella, E. coli and select strains of Clostridia, making it a potential new direct-fed microbial that could be used in Kemin's intestinal health portfolio. The biggest in vitro advantages with D3 over CLOSTAT® and ENTEROSURE® have been seen with Salmonella and E. coli.

In this trial, pigs received a strong E. coli challenge which led to high overall mortality. Mortality was highest in pigs in the positive control (Mecadox) group. Mortality in the positive control was higher than the negative control, which is unusual, indicating that the natural challenge was very high that even the antibiotic was not able to help in this scenario. It was assumed by the farm veterinarian that along with the E. coli infection, the piglets had a viral co-infection, possible a rotavirus infection but this claim was not verified. LBP levels further support the possibility of multiple enteric challenges because LPS, measured by serum LBP, were highest in the NC, but the NC had among the lowest mortalities. That provides some explanation for the mortality being high even in the antibiotic group. Even under these circumstances, D3, fed at 1E5 CFU/g had the lowest mortality and was significantly lower than the positive control, indicating D3 may help reduce mortality in pigs challenged with E coli outbreaks.

### Example 4: Effect of Supplementing Bacillus velezensis, D3, on the Health and Feedlot Performance of Calf Fed Steers and Heifers

### Study Design

This study was completed at Bartonek Farms Feedlot in Olmitz, KS. Cattle (759 head) were randomly assigned to one of two groups, T1 or T2. Cattle in T2 (D3 supplemented) received the same ration as T1 (control) with the addition of the D3 supplement. Cattle were fed twice daily. The duration of feeding was from Day 0 (first day of study) to termination. The study was a randomized complete block design with each pen as the experimental unit. The D3 supplement was Tex Bio batch FEP-D3-2310001; 2.73 E8 CFU/g, with a supplement feed rate of 10 grams per head per day (g/h/d). This equated to a daily dose of 2.73 E9 CFU/h/d. The supplement contained calcium carbonate free-flow (RM01029 Lot 2306113714) as a carrier. The supplement was added to feed in a Roto-Mix truck and mixed for five minutes before feeding.

### Body Weights

Esfeld heifers (n=198) in the control group had an average initial weight of 721.7 pounds, which was 5.5 pounds heaver than the average weight of cattle in the D3 supplemented group (n=297; 727.2 pounds; p=0.2281). The final average weight of Esfeld heifers in the control group was 839.3 pounds or 12.9 pounds less than cattle fed D3 (852.2 pounds; p=0.0079). See Table 13.

Turner heifers (n=18) in the control group had an average initial weight of 500.0 pounds, which was 8.9 pounds heaver than the average weight of cattle in the D3 supplemented group (n=100; 491.1 pounds; p=0.5518). The final average weight of Turner heifers in the control group was 726.6 pounds or 44.3 pounds less than cattle fed D3 (770.9 pounds; p=0.00136). See Table 13.

Turner steers (n=46) in the control group had an average initial weight of 505.9 pounds, which was 35.5 pounds less than the average weight of cattle in the D3 supplemented group (n=100; 541.2 pounds; p=0.0001). The final average weight of Turner steers in the control group was 734.9 pounds or 108.9 pounds less than cattle fed D3 (843.8 pounds; p=0.0001). See Table 13.

TABLE 13
Average body weights of Esfeld and Turner cattle. Values in a group with
different letter (a-b) are significatnly different (p ≤ 0.05).

| | | | Average | | Initial | Average | | Final | Corrected |
|---|---|---|---|---|---|---|---|---|---|
| | | | Initial | | Weight | Final | | Weight | Weight |
| | | Sample | Weight | | Difference | Weight | | Difference | Difference |
| Breed | Group | Size (n) | (lb.) | p-value | (lb.) | (lb.) | p-value | (lb.) | (lb.)* |
| Esfeld | Control | 198 | 721.7a | 0.2281 | D3: +5.5 | 839.3b | 0.0079 | D3: +12.9 | D3: +7.4 |
| Heifers | D3 | 297 | 727.2a | | | 852.2a | | | |
| Turner | Control | 18 | 500.0a | 0.5518 | Control: +8.9 | 726.6a | 0.0136 | D3: +44.3 | D3: +53.2 |
| Heifers | D3 | 100 | 491.1a | | | 770.9b | | | |
| Turner | Control | 46 | 505.9a | 0.0001 | D3: +35.3 | 734.9a | 0.0001 | D3: +108.9 | D3: +73.6 |
| Steers | D3 | 100 | 541.2b | | | 843.8b | | | |

*Corrected for intial weight difference.

Combined Turner cattle (n=64) in the control group had an average initial weight of 504.2 pounds which was 12 pounds less than the average weight of Turner cattle in the D3 supplemented group (n=200; 516.2 pounds; p=0.1348). The final average weight of Turner cattle in the control group was 732.6 pounds or 74.7 pounds less than the average weight of Turner cattle fed D3 (807.3 pounds; p=<0.0001). See Table 14.

TABLE 14
Combine average body weights of Turner heifers and steers. Values with
different letter (a-b) are significantly different (p ≤ 0.05).

| | | | Average | | Initial | Average | | Final | Corrected |
|---|---|---|---|---|---|---|---|---|---|
| | | | Initial | | Weight | Final | | Weight | Weight |
| | | Sample | Weight | | Difference | Weight | | Difference | Difference |
| Breed | Group | Size (n) | (lb.) | p-value | (lb.) | (lb.) | p-value | (lb.) | (lb.)* |
| Combined | Control | 64 | 504.2a | 0.1348 | D3: +12.0 | 732.6a | <0.0001 | D3: +74.7 | D3: +62.7 |
| Turner | D3 | 200 | 516.2a | | | 807.3b | | | |
| Cattle | | | | | | | | | |

*Corrected for intial weight difference.

Combined Turner and Esfeld heifer (n=613) average initial weight was 703.2 pounds in the control group (n=216) and was significantly higher (p<0.0001) than the average weight of cattle in the group fed D3 (n=397; 667.7 pounds). The final average weight of all heifers in the control group was 829.9 pounds or 1.8 pounds less than cattle fed D3 (831.7 pounds; p=0.7430). See Table 15.

TABLE 15
Combine average body weights of all heifers. Values with different letter (a-b)
are significantly different (p ≤ 0.05).

| | | | Average | | Initial | Average | | Final | Corrected |
|---|---|---|---|---|---|---|---|---|---|
| | | | Initial | | Weight | Final | | Weight | Weight |
| | | Sample | Weight | | Difference | Weight | | Difference | Difference |
| Breed | Group | Size (n) | (lb.) | p-value | (lb.) | (lb.) | p-value | (lb.) | (lb.)* |
| Combined | Control | 64 | 703.2a | <0.0001 | D3: −35.5 | 829.9a | 0.7430 | D3: +1.8 | D3: +37.3 |
| heifers | D3 | 200 | 667.7b | | | 831.7b | | | |

*Corrected for intial weight difference.

Esfeld heifers had a 16.3 and 17.2 percent average increase in bodyweight for the control and D3 supplemented groups respectively. Turner heifers had a 45.3 and 57.0 average percent increase in bodyweight for the control and D3 supplemented groups respectively. Turner steers had a 45.3 and 55.9 average percent increase in bodyweight for the control and D3 supplemented groups respectively. Combined Turner cattle had a 45.3 and 56.4 average percent increase in bodyweight for the control and D3 supplemented groups respectively. Combined heifers had a 18.0 and 21.9 percent average increase in bodyweight for the control and D3 supplemented groups respectively. See Table 16.

TABLE 16
Average percent weight gain.

| | Intial | Final | Percent Gain | Difference (%) |
|---|---|---|---|---|
| Esfeld_Control | 721.7 | 839.3 | 16.3 | D3: + 0.9 |
| Esfeld_D3 | 727.2 | 852.2 | 17.2 | |
| Turner Heifer_Control | 500 | 726.3 | 45.3 | D3: + 11.7 |
| Turner Heifer_D3 | 491.1 | 770.9 | 57.0 | |
| Turner Steer_Control | 505.9 | 734.9 | 45.3 | D3: + 10.6 |
| Turner Steer_D3 | 541.2 | 843.8 | 55.9 | |
| Combined | 504.2 | 732.6 | 45.3 | D3: + 11.1 |
| Turner_Control | | | | |
| Combined Turner_D3 | 516.2 | 807.3 | 56.4 | |
| Combined | 703.2 | 829.9 | 18.0 | D3: + 3.9 |
| heifers_Control | | | | |
| Combined heifers_D3 | 667.7 | 831.7 | 21.9 | |

Cattle with bovine raspatory disease (BRD) were treated with Nuflor, Exceded, Micotil or a combination of the treatments. See Table 17.

TABLE 17
Incidence of bovine respitory disease (BRD) treatment and percent of
animals treated per group.

| | | Sample | Treatment Incidence (%) | | |
|---|---|---|---|---|---|
| Breed | Group | Size (n) | 1 | 2 | 3 |
| Esfeld | Control | 198 | 19 (9.5) | 5 (2.5) | 8 (4) |
| Heifer | D3 | 297 | 14 (4.7) | 5 (1.7) | 3 (1.0) |
| Turner | Control | 18 | 1 (5.6) | 2 (11.1) | 2 (11.1) |
| Heifer | D3 | 100 | 11 (11.0) | 5 (5.0) | 2 (2.0) |
| Turner | Control | 46 | 5 (10.9) | 2 (4.3) | 2 (4.3) |
| Steer | D3 | 100 | 9 (9.0) | 3 (3.0) | 1 (1.0) |

Overall, Turner cattle and Esfeld heifers fed D3 showed increased average weight gain when compared to cattle in their respective control groups. These data suggest that D3 acts as a growth promotor. Incidence of BRD in cattle may also be reduced when fed D3.

### Example 5: Supplementation of Bacillus velezensis, D3, Plus Clove Oil

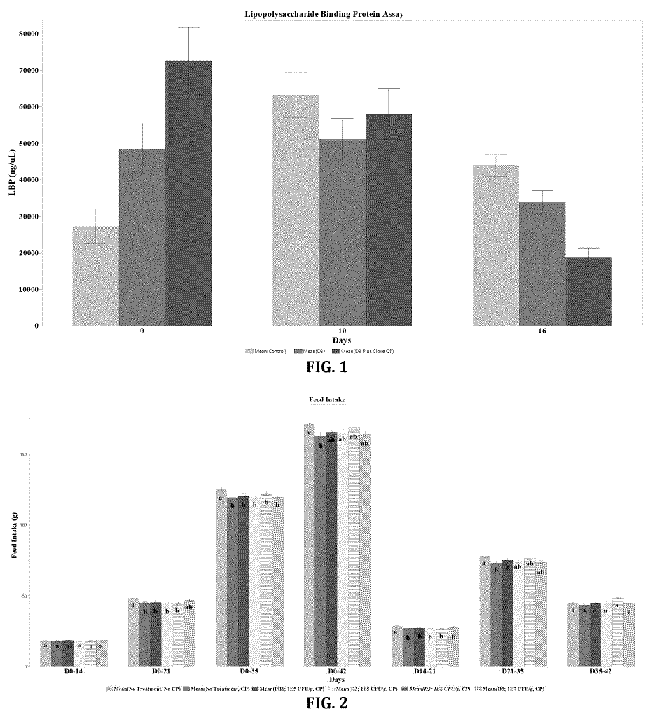

In a USDA trial, D3 and D3 plus clove oil was feed to mixed breed calves. D3 (1.3E9 CFU/head/day) or D3 (1.3E9 CFU/head/day) plus clove oil (1.4 g/head/day) supplemented calves were compared to calves that received no treatment (control). Serum samples collected on days 0, 10 and 16 showed that lipopolysaccharide binding protein (LBP) levels decreased in calves supplemented with D3 or D3 plus clove oil, indicating the level of lipopolysaccharide (LPS) challenge in the blood was reduced with supplementation of D3 or D3 with clove oil. See FIG. 1.

### Example 6: Bacillus velezensis D3 Necrotic Enteritis Trial Efficacy

The objective of this study was to evaluate the ability of D3 to improve performance parameters after clostridial challenge in coccidiosis challenged broiler chickens at various doses. CLOSTAT®, B. subtilis PB6 was included as a positive control.

### Materials and Methods

Experimental probiotic. The CFU/g in each treatment provided to the test facility is shown in Table 18. Each experimental treatment was assayed. In brief, 1:9 dilutions were prepared in 0.85% sterile saline, vortexed, and further diluted 1:9. This dilution was heated to 80° C. for 10 min and immediately cooled in a cold-water bath for 2 min. Further dilutions were made to the endpoint, plated in duplicate on 3M aerobic count (AC) petrifilms (St. Paul, MN), and incubated overnight at 37° C.

Feed sample analysis. Post-pelleted feed samples were tested to verify the targeted CFU/g in feed. Feed samples were tested. Briefly, triplicate 1:9 dilutions were prepared in 0.85% sterile saline, stomached in a Seward Stomacher 80 (Seward, Islandia, NY) for 30 seconds at medium speed, and further diluted 1:9. This dilution (1:100) was heated to 80° C. for 10 min and immediately cooled in a cold-water bath for 2 min. Further dilutions were made to the endpoint, plated in duplicate on 3M AC films, and incubated overnight at 37° C.

Experimental facility. The floor pen unit was FM Unit #1 located at 96 Roquemore Rd., Athens, GA. The experimental house is divided into pens of equal size, arranged along a central aisle. Birds were placed in 78 pens each having an area of 5×10=50 ft². All pens had approximately 4 inches of built-up litter with a coating of fresh pine shavings. Poultry production in the United States routinely uses built-up litter. This study is designed to mimic, as close as possible, commercial conditions, so built-up litter was used. The initial stocking density, after subtracting for equipment, was ~0.91 ft²/bird or 46 birds per pen. Each pen has 5-feet-high side walls with the bottom 1½ feet being solid wood to prevent bird migration. There were 13 pens and 598 birds per treatment.

Design of the necrotic enteritis challenge trial. Day of hatch Cobb 500 male chicks were obtained from Cobb Vantress hatchery, Cleveland, GA; 3588 chicks were allocated to the study. Birds were handled according to Institutional Animal Care and Use Committee (IACUC) guidelines and monitored by an SPFR veterinarian. Accountabilities of all test animals and any extra birds were recorded on the animal disposition form. The breeder flock history and vaccination record at the hatchery were recorded. Only healthy appearing chicks were used in the study. All birds were spray vaccinated with a commercial coccidia vaccine (Coccivac-B52) at the recommended use level prior to placement in pens. At study initiation, 46 chicks were allocated to each treatment pen by blocks. Chicks were allocated at random by block to each pen and carefully counted to assure the correct numbers of birds were placed into each experimental pen. No birds were replaced during the study. Bird weights (kg) by pen were recorded at study initiation (day 0) and on days 14, 21, 35, and 42.

Feed and water. Feed and water were provided ad libitum from day 0 through 42 of the study. Broiler diets were fed as crumbles (starter feed, d0-21) or as pellets (grower; d21-35) and finisher (d35-42). Feed formulations for this study consisted of unmedicated commercial-type broiler starter diets compounded with commonly used United States feedstuffs representative of local formulations calculated to meet or exceed National Research Council (NRC) standards. No antibiotics were added to any feed. Treatments were prepared from a basal starter feed, as depicted in Table 18. Treatment feed was mixed at the Southern Poultry Feed and Research feed mill and pelleted in a California Pellet Mill at 80° C.

TABLE 18
Feed treatment types, challenge, and inclusion levels.

| | | Coccidia | Clostridium | |
|---|---|---|---|---|
| No. | Treatment | Challenge | perfringens | Pens/Trt |
| 1 | No Treatment, No CP | DOT 14 | No | 13 |
| 2 | No Treatment, CP | DOT 14 | DOT 19, 20, and 21 | 13 |
| 3 | PB6 (1E5 CFU/g) | DOT 14 | DOT 19, 20, and 21 | 13 |
| 4 | D3 (1E5 CFU/g) | DOT 14 | DOT 19, 20, and 21 | 13 |
| 5 | D3 (1E6 CFU/g) | DOT 14 | DOT 19, 20, and 21 | 13 |
| 6 | D3 (1E7 CFU/g) | DOT 14 | DOT 19, 20, and 21 | 13 |

CP: C. perfringens challenge; Trt: treatment; DOT: Day of trial

Disease induction. On day of trial (DOT) 14, all birds were orally inoculated with ~5,000 oocysts of E. maxima per procedures described in a Southern Poultry Feed and Research (SPFR) SOP. Starting on DOT 19, all birds, except Treatment 1, were given 1 mL of a broth culture of Clostridia perfringens (CP) containing ~1E8 CFU/mL. The CP is an isolate from a clinical case of necrotic enteritis (NE). It is both alpha toxin and net B toxin positive. The birds were administered a fresh broth culture once daily for 3 days (on DOT 19, 20, and 21).

Monitoring. All birds were monitored for general flock condition. In addition, temperature, lighting, water, feed, litter condition, and any unanticipated house conditions/events were noted. Findings were documented twice daily during the regular working hours with one observation recorded on the final day of the study.

Performance and mortality parameters. The birds and feed were weighed by pen on d0, 14, 21, 35, and 42. Means for pen weight gain, feed consumption, and feed conversion ratio (FCR) were then calculated (d0-14, 0-21, 0-35, 0-42, 14-21, 21-35, and 35-42). FCR was adjusted to account for mortality occurring during the study.

Necrotic enteritis intestinal lesion score. On DOT 21 and 35, three birds from each pen were euthanized, weighed, and examined for the degree of presence of NE lesions. The scoring was based on a 0 to 3 score, with 0 being normal and 3 being the most severe.
Lesion score 0=Normal
Lesion score 1=Slight mucus covering small intestine
Lesion score 2=Necrotic small intestine mucosa
Lesion score 3=Sloughed and bloody small intestine mucosa and contents

Statistical analysis. Southern Poultry Feed and Research (SPFR) analyzed the trial data with STATIX, using least-squared differences for analysis of means (P<0.05). The data were further evaluated using the JMP software package (version 17.0.0), using analysis of variance (ANOVA) with comparison of means using Turkey t test (P<0.05). Outliers were removed.

### Results

Feed was provided as crumbles (starter) or pellets (grower/finisher), and post-pelleted feed samples were tested to verify the level of D3 and PB6 (CLOSTAT®) delivered to the test birds. Treatments 1 and 2 did not include a probiotic and were analyzed for background counts only (Table 19). Treatment 3 contained PB6 at a target level of 1E5 CFU/g. Treatments 4, 5, and 6 contained D3 at target levels of 1E5, 1E6, and 1E7 CFU/g of feed, respectively. Expected treatment counts (Table 18) were higher than target treatment counts due to the treatment prep counts (CFU/g) being higher than target (Table 14). Over formulating of treatment preps is by design due to expected losses of probiotics during the pelleting process. Background counts were 1.4 to 1.5E4 CFU/g (Table 19). Treatment 3 and 4 counts were in line with expected values of 1E5 CFU/g for all feed mixes (starter, grower finisher). Treatment 5 counts were ~0.3 log below the expected count of 1E6 CFU/g in both starter and finisher feed samples. Treatment 6 counts were 0.5, 0.4, and 0.4 log below expected values of 1E7 CFU/g in starter, grower, and finisher samples respectively.

TABLE 19
Counts (CFU/g) in post-pelleted feed samples. (n = 3)

| | | | | | Actual |
|---|---|---|---|---|---|
| | | | Target | Expected | Counts |
| Treatment | Feed | Probiotic | (CFU/g) | (CFU/g) | (CFU/g) |
| 1 and 2 | Starter | N/A | N/A | N/A | 1.55E4 |
| 3 | Starter | PB6 | 1E5 | 2.43E5 | 2.15E5 |
| 4 | Starter | D3 | 1E5 | 1.68E5 | 1.26E5 |
| 5 | Starter | D3 | 1E6 | 2.33E6 | 6.15E5 |
| 6 | Starter | D3 | 1E7 | 1.23E7 | 4.93E6 |
| 1 and 2 | Grower | N/A | N/A | N/A | 1.47E4 |
| 3 | Grower | PB6 | 1E5 | 2.43E5 | 1.99E5 |
| 4 | Grower | D3 | 1E5 | 1.68E5 | 1.17E5 |
| 5 | Grower | D3 | 1E6 | 2.33E6 | 1.14E6 |
| 6 | Grower | D3 | 1E7 | 1.23E7 | 5.67E6 |
| 1 and 2 | Finisher | N/A | N/A | N/A | 1.38E4 |
| 3 | Finisher | PB6 | 1E5 | 2.43E5 | 2.12E5 |
| 4 | Finisher | D3 | 1E5 | 1.68E5 | 1.16E5 |
| 5 | Finisher | D3 | 1E6 | 2.33E6 | 7.37E5 |
| 6 | Finisher | D3 | 1E7 | 1.23E7 | 6.05E6 |

N/A: Not applicable.

Overall mortality was highest in the challenged control (no treatment, CP) group at 9.1% and was significantly higher (p<0.05) than the unchallenged control (no treatment, no CP) at 4.6% and group 6 (D3; 1E6 CFU/g) at 4.5%. Birds fed PB6 (Treatment 3), and birds fed D3 (Treatments 4, 5, and 6) had mortalities of 5.6, 5.4, 4.5, and 5.2%, respectively, and were statistically the same (p<0.05). Birds fed D3 at 1E6 CFU/g (Treatment 5) had the lowest mortality at 4.5%, which was significantly lower (p<0.05) than the challenged control (no additive, CP) but not statistically (p<0.05) different from the other treatment groups (Table 20).

TABLE 20
Average percent mortality of birds for days 0-42.

| | | Percent |
|---|---|---|
| No. | Treatment | Mortality ± SE |
| 1 | No Treatment, no CP | 4.6 ± 0.9^{b} |
| 2 | No Treatment, CP | 9.1 ± 1.0ª |
| 3 | PB6 (1E5 CFU/g) | 5.6 ± 0.9^{ab} |
| 4 | D3 (1E5 CFU/g) | 5.4 ± 1.0^{ab} |
| 5 | D3 (1E6 CFU/g) | 4.5 ± 0.9^{b} |
| 6 | D3 (1E7 CFU/g) | 5.2 ± 1.0^{ab} |

SE: standard error; CP: C. perfringens
Mean ± SE. Values not connected by the same letter^{a-b} are significantly different (p < 0.05).

All birds found dead were necropsied. NE mortality (d0-42), or death attributed to necrotic enteritis lesions in the intestine, was highest in the challenged control (no additive, CP) group at a mortality rate of 4.3% and was significantly higher than groups 1, 5, and 6 (p<0.05). Groups 3, 4, 5, and 6 had NE mortality rates of 2.3, 2.4, 0.8, and 2.0%, respectively, and did not significantly differ (p<0.05). The unchallenged control (no additive, no CP) group had no NE mortality and was significantly (p<0.05) lower than all groups except group 5 (D3 1E6 CFU/g). NE mortality results are summarized in Table 21.

TABLE 21
Average percent necrotic enteritis mortality (d0-42) of broilers
in all treatment groups.

| | | Percent NE |
|---|---|---|
| No. | Treatment | mortality ± SE |
| 1 | No Treatment, no CP | 0.0 ± 0.4^{c} |
| 2 | No Treatment, CP | 4.3 ± 0.6ª |
| 3 | PB6 (1E5 CFU/g) | 2.3 ± 0.4^{ab} |
| 4 | D3 (1E5 CFU/g) | 2.4 ± 0.5^{ab} |
| 5 | D3 (1E6 CFU/g) | 0.8 ± 0.4^{bc} |
| 6 | D3 (1E7 CFU/g) | 2.0 ± 0.4^{b} |

NE: Necrotic enteritis; SE: standard error; CP: C. perfringens
All groups' sans treatment 1 (no treatment, no CP) were challenged with C. perfringens on d19, 20 and 21. Mean ± SE. Values not connected the by same letter are significantly different (p < 0.05)

On DOT 21 and 35, three birds from each pen were sacrificed, weighed, and examined for the degree of NE lesions. The NE day 21 lesion score was highest in the challenged control (no additive, CP) group with a score of 1.5 and was significantly higher than all other groups (p<0.05). Groups 3, 4, 5, and 6 had day 21 lesion scores of 0.7, 0.6, 0.8, and 0.7, respectively, and did not significantly differ (p<0.05) but were significantly higher (p<0.05) than the unchallenged control (no additive, no CP). Necrotic enteritis day 35 lesion scores did not significantly (p<0.05) differ. NE day 21 and day 35 lesion scores are summarized in Table 22.

TABLE 22
Average day 21 and day 35 NE lesion scores of broilers in
all treatment groups.

| | Day 21 average NE | Day 35 average NE |
|---|---|---|
| Treatment | lesion score ± SE | lesion score ± SE |
| No Treatment, no CP | 0.0 ± 0.1ª | 0.0 ± 0.1 |
| No Treatment, CP | 1.5 ± 0.1^{c} | 0.3 ± 0.1 |
| PB6 (1E5 CFU/g) | 0.7 ± 0.1^{b} | 0.2 ± 0.2 |
| D3 (1E5 CFU/g) | 0.6 ± 0.1^{b} | 0.2 ± 0.1 |
| D3 (1E6 CFU/g) | 0.8 ± 0.1^{b} | 0.2 ± 0.1 |
| D3 (1E7 CFU/g) | 0.7 ± 0.1^{b} | 0.2 ± 0.1 |

NE: Necrotic enteritis; SE: standard error; CP: C. perfringens
All groups sans treatment 1 (no treatment, no CP) were challenged with C. perfringens on d19, 20, and 21. Mean ± SE. Values not connected the by same letter are significantly different (p < 0.05).

No differences in feed intake were seen for days 0-14, 0-21, 0-35, 0-42, and 14-21. (FIG. 2 and Table 23; p<0.05). The unchallenged control (no treatment, no CP) group had significantly higher feed intake than the challenged control (no treatment, CP) on days 21-35 (FIG. 2 and Table 23; p<0.05), and group 5 (D3; 1E6 CFU/g) feed intake was significantly higher than the challenged control (no treatment, CP) on days 35-42 (FIG. 2 and Table 23; p<0.05). No differences were seen among the treated groups (groups 3-6; FIG. 2 and Table 23). Values for feed intake are shown in FIG. 2 and Table 23.

TABLE 23
Feed intake at 0-14 d, 0-21 d, 0-35 d, 0-42 d, 14-21 d, 21-35 d, and 35-42 d of
broilers in all treatment groups.
Feed Intake (g)

| Treatment No. | Treatment | 0-14 d | 0-21 d | 0-35 d | 0-42 d | 14-21 d | 21-35 d | 35-42 d |
|---|---|---|---|---|---|---|---|---|
| 1 | No Treatment, No CP | 18.34 | 48.41 | 125.67 | 171.84 | 29.31 | 78.40^{a} | 45.51^{ab} |
| 2 | No Treatment, CP | 18.20 | 45.46 | 119.22 | 163.25 | 27.26 | 73.31^{b} | 43.57^{b} |
| 3 | PB6 ( 1E5 CFU/g) | 18.35 | 45.61 | 120.65 | 165.49 | 27.26 | 75.04^{ab} | 44.83^{ab} |
| 4 | D3 (1E5 CFU/g) | 18.22 | 45.54 | 120.55 | 165.84 | 27.32 | 75.01^{ab} | 45.29^{ab} |
| 5 | D3 (1E6 CFU/g) | 18.28 | 45.35 | 122.32 | 169.82 | 27.07 | 76.97^{ab} | 48.60^{a} |
| 6 | D3 (1E7 CFU/g) | 18.89 | 47.00 | 119.22 | 164.41 | 27.78 | 74.04^{ab} | 44.75^{ab} |

All treatments sans treatment 1 (no treatment, no CP) were challenged with C. perfringens on d19, 20, and 21.
Mean ± SE.
*Values with different superscript letters^{a-b} indicate differences in means (P < 0.05).
CP: C. perfringens challenge.
SE: standard error.

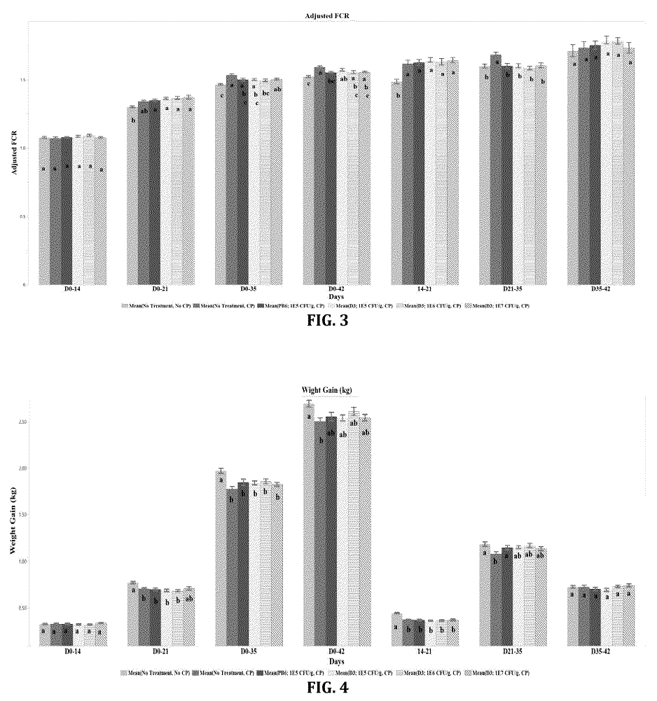

No differences in adjusted FCR were seen for days 0-14 and 35-42. (FIG. 3 and Table 24; p<0.05). For days 0-21, the unchallenged control (no treatment, no CP) group had a significantly (P<0.05) lower adjusted FCR than all groups sans the challenged control (no treatment, CP; FIG. 3 and Table 24; p<0.05). For days 0-35, the unchallenged control (no treatment, no CP) group's adjusted FCR was significantly (P<0.05) lower than the challenged control (no treatment, CP) and treatment 6 (D3; 1E7 CFU/g). Also, for days 0-35, Group 5 (D3; 1E6 CFU/g) adjusted FCR was significantly (P<0.05) lower than the challenged control (no treatment, CP). For days 0-42, the unchallenged control (no treatment, no CP) had a lower adjusted FCR than the challenged control (no treatment, CP) and treatment 4 (D3; 1E5 CFU/g), and the challenged control (no treatment, CP) adjusted FCR was significantly higher (P<0.05) than treatment 3 (PB6; 1E5 CFU/g). For days 14-21, the unchallenged control (no treatment, no CP) was significantly (P<0.05) lower than all other groups, and for days 21-35, the challenged control (no treatment, CP) adjusted FCR was higher than all other groups. In all time periods, no differences were seen among the challenged treated groups. Values for adjusted FCR are shown in FIG. 3 and Table 24.

TABLE 24
Adjusted FCR at 0-14 d, 0-21 d, 0-35 d, 0-42 d, 14-21 d, 21-35 d, and 35-42 d of
broilers in all treatment groups.
Adjusted FCR

| Adjusted FCR | | | | | | | | |
|---|---|---|---|---|---|---|---|---|
| Treatment No. | Treatment | 0-14 d | 0-21 d | 0-35 d | 0-42 d | 14-21 d | 21-35 d | 35-42 d |
| 1 | No Treatment, No CP | 1.08 | 1.30^{b} | 1.47^{c} | 1.52^{c} | 1.49^{b} | 1.60^{b} | 1.71 |
| 2 | No Treatment, CP | 1.07 | 1.34^{ab} | 1.54^{a} | 1.59^{a} | 1.62^{a} | 1.68^{a} | 1.73 |
| 3 | PB6 (1E5 CFU/g) | 1.08 | 1.35^{a} | 1.50^{abc} | 1.55^{bc} | 1.63^{a} | 1.60^{b} | 1.75 |
| 4 | D3 (1E5 CFU/g) | 1.09 | 1.36^{a} | 1.50^{abc} | 1.58^{ab} | 1.65^{a} | 1.60^{b} | 1.79 |
| 5 | D3 (1E6 CFU/g) | 1.09 | 1.37^{a} | 1.50^{bc} | 1.56^{abc} | 1.63^{a} | 1.59^{b} | 1.78 |
| 6 | D3 (1E7 CFU/g) | 1.08 | 1.36^{a} | 1.51^{ab} | 1.56^{abc} | 1.64^{a} | 1.60^{b} | 1.73 |

All treatments sans treatment 1 (no treatment, no CP) were challenged with C. perfringens on d19, 20, and 21.
Mean ± SE.
*Values with different superscript letters^{a-c} indicate differences in means (P < 0.05).
CP: C. perfringens challenge.
SE: standard error.

There were no differences in body weight gain for days 0-14 and 35-42 (FIG. 4 and Table 25; p>0.05). For days 0-21, there were significant (P<0.05) differences in weight gain between the unchallenged control (no treatment, no CP) and all other groups except for group 6 (D3; 1E7 CFU/g). For days 0-35 and 14-21, weight gain was significantly (P<0.05) higher in the unchallenged (no treatment, no CP) group. For days 0-42, the unchallenged control (no treatment, no CP) was significantly (P<0.05) higher than the challenged control (no treatment, CP). For days 21-35, the challenged control (no treatment, CP) was significantly (P<0.05) lower than the unchallenged control (no treatment, no CP) and treatment 3 (PB6; 1E5 CFU/g). No differences were seen among treated challenged groups (FIG. 4 and Table 25). Values for weight gain are shown in FIG. 4 and Table 25.

TABLE 25
Weight gain at 0-14 d, 0-21 d, 0-35 d, 0-42 d, 14-21 d, 21-35 d, and 35-42 d of
broilers in all treatment groups.
Weight Gain (kg)

| Treatment No. | Treatment | 0-14 d | 0-21 d | 0-35 d | 0-42 d | 14-21 d | 21-35 d | 35-42 d |
|---|---|---|---|---|---|---|---|---|
| 1 | No Treatment, No CP | 0.33 | 0.77^{a} | 1.97^{b} | 2.69^{a} | 0.45^{a} | 1.19^{a} | 0.73 |
| 2 | No Treatment, CP | 0.33 | 0.71^{b} | 1.78^{a} | 2.50^{b} | 0.38^{b} | 1.08^{b} | 0.73 |
| 3 | PB6 (1E5 CFU/g) | 0.33 | 0.70^{b} | 1.85^{b} | 2.55^{ab} | 0.37^{b} | 1.15^{ab} | 0.70 |
| 4 | D3 (1E5 CFU/g) | 0.32 | 0.69^{b} | 1.84^{b} | 2.54^{ab} | 0.37^{b} | 1.15^{ab} | 0.70 |
| 5 | D3 (1E6 CFU/g) | 0.33 | 0.69^{b} | 1.86^{b} | 2.61^{ab} | 0.37^{b} | 1.17^{ab} | 0.73 |
| 6 | D3 (1E7 CFU/g) | 0.34 | 0.71^{ab} | 1.83^{b} | 2.54^{ab} | 0.37^{b} | 1.14^{ab} | 0.74 |

All treatments sans treatment 1 (no treatment, no CP)
were challenged with C. perfringens on d19, 20, and 21.
Mean ± SE.
*Values with different superscript letters^{a-b} indicate differences in means (P < 0.05).
CP: C. perfringens challenge.
SE: standard error.

### Discussion

Bacillus velezensis D3 has shown superior in vitro efficacy against Salmonella, E. coli and select strains of Clostridia, making it a novel direct-fed microbial. While performance differences were not seen between D3 and PB6, mortality rate was significantly (p<0.05) lower with D3 at 1E6 CFU/g when compared to the challenged (no treatment, CP) control, making treatment 5 the only treatment to show a significant reduction in mortality. A 51% lower mortality rate in group 5 (4.5%; D3; 1E6 CFU/g) compared to the challenged control (9.1%, no treatment, CP) could mean greater profits for producers, so this finding is meaningful. No differences in NE lesion score were seen among the treatments, suggesting D3 is just as effective as CLOSTAT® at preventing NE.

### Example 7: Steamed Flaked Corn D3 Recovery

Materials and Methods. D3 lot FEP-D3-2401001 spores (2.97E9 CFU/g) on a calcium carbonate carrier was added to hot steamed flaked corn ranging in temperature from 149-152° F. (65-67° C.) and thoroughly mixed to ensure the spores were evenly distributed. A sample allowed to cool to ambient (65° F.; 18° C.) with D3 and an ambient (70° F.; 21° C.) control sample without D3 was also prepared. Samples (1 gallon) of each condition were shipped to KANA R and D for enumeration. To enumerate, 10-fold dilutions in 0.85% saline (saline) were prepared, vortexed, and further diluted 10-fold to Oct. 3, 2010-5 with saline. Dilution of samples with D3 (10-5) and without D3 (10-3) were plated on Neogen aerobic count (AC) Petri films (Lansing, MI) and incubated aerobically overnight at 37° C. Films were enumerated 18-24 hours after plating using a 3M Petri film reader or by manually counting colonies.

Results: The addition of D3 (10 g; 2.97E9 CFU/g) to 10 kg of steamed flaked corn was calculated to contain 2.97E6 CFU/g. Actual recoveries ranged from 1.08E6 to 1.28E6 CFU from hot steamed flaked corn, which was lower than the 1.68E6 CFU/g recovery from ambient temperature steamed flaked corn containing D3. Hot steamed flaked corn caused a 0.1 to 0.2 log reduction compared to the ambient temperature steamed flaked corn sample, demonstrating D3s heat tolerance. Steamed flaked corn ambient background counts (no added D3) of 2.97E4 were in the range of total aerobic counts seen in feed matrices. See Table 26.

TABLE 26

| D3 recovery from hot streamed flaked, D3 recovery from ambient | |
|---|---|
| temperature streamed flaked corn and total aerobic count | |
| recovery form streamed flaked corn without added D3. | |
| Sample ID | Counts (CFU/g) |
| Control; 70° F., no D3 | 2.97E4* |
| D3 ambient; 65° F. | 1.68E6 |
| D3 hot; 150° F. | 1.28E6 |
| D3 hot; 152° F. | 1.35E6 |
| D3 hot; 149º F. | 1.08E6 |

*Total Aerobic Counts

### Example 8: D3 Stability

Materials and Methods. D3 lot FEP-D3-2401001 spores were stored in conical tubes (50 mL) at ambient and 40° C. for 12 months to assess stability. Humidity was not controlled for either storage condition. Samples were tested at 0, 3, 6, 9, and 12 months. To enumerate, 10-fold dilutions in 0.85% saline (saline) were prepared, vortexed, and further diluted 10-fold to 10-9 with saline. The second dilutions (10-2) were heated to 80° C. for 10 minutes and immediately cooled in a cold-water bath for two minutes to kill vegetative cells. Dilutions (10-8 and 10-9) were plated on Neogen aerobic count (AC) Petri films (Lansing, MI) and incubated aerobically overnight at 37° C. Films were enumerated 18-24 hours after plating using a 3M Petri film reader or by manually counting colonies.

Results: Initial spore counts (TO; 8.82E10 CFU/g) decreased to 5.83E10 and 6.33E10 CFU/g under ambient and accelerated (40° C.) conditions, respectively. Losses were less than 0.5-log under both conditions which demonstrated good stability. See Table 27.

TABLE 27
D3 12-month stability at ambient and accelerated (40° C.) storage conditions.
D3 Lot FEP-D3-2401001 Stability

| Storage Condition | 0 | 3 | 6 | 9 | 12 |
|---|---|---|---|---|---|
| Ambient Counts | 8.82E+10 | 8.12E+10 | 6.00E+10 | 7.25E+10 | 5.83E+10 |
| (CFU/g) | | | | | |
| Accelerated (40° C.) | 8.82E+10 | 8.32E+10 | 6.55E+10 | 6.90E+10 | 6.33E+10 |
| Counts (CFU/g) | | | | | |

It should be appreciated that minor dosage and formulation modifications of the composition and the ranges expressed herein may be made and still come within the scope and spirit of the present invention.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplish at least all of the intended objectives.

1. An isolated Bacillus velezensis strain having deposit accession number PTA-127643, or a mutant thereof having at least 95% sequence identity.

2. A direct-fed microbial composition comprising an isolated Bacillus velezensis strain having deposit accession number PTA-127643, or a mutant thereof.

3. The composition of claim 2, wherein the composition is an animal feed additive.

4. The animal feed according to claim 3, wherein the Bacillus velezensis PTA-127643 is present with a bacterial count between about 1×10³ and 1×10¹⁴ CFU/kg of animal feed.

5. The animal feed or animal feed additive according to claim 3, further comprising a carrier.

6. The animal feed or animal feed additive according to claim 5, wherein the carrier comprises one or more of the following compounds: water, silica, glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, maltodextrin, white oil, corn cob meal, rice bran, glucose, sucrose, sorbitol, lactose, wheat flour, wheat bran, corn gluten meal, starch and cellulose.

7. The animal feed or animal feed additive of claim 3, further comprising at least one enzyme, prebiotic, probiotic, supplement, or milk replacer.

8. The composition of claim 2, wherein Bacillus velezensis PTA-127643 is present as spores.

9. A method of inhibiting or reducing pathogen growth in animal feed comprising adding a composition comprising Bacillus velezensis PTA-127643 to the animal feed.

10. The method of claim 9, further comprising the step of administering the composition to an animal, wherein the administration improves one or more performance parameter of the animal.

11. The method of claim 9, wherein the pathogen is selected from the group consisting of E. coli, S. Typhimurium, S. Litchfield, S. Enterica, S. Montevideo, S. Panama, S. Schwarzengrund, S. aureus, E. gallinarium, S. agalactiae, S. enteritidis, S. Senftenberg, R. equi, S. equi, S. Derby, E. cecorum, C. perfringens, C. difficile, C. septicum, and S. suis.

12. The method of claim 10, wherein the one or more performance parameter is selected from the group consisting of body weight gain, European production efficiency factor, feed conversion ratio, animal health, and egg yield.

13. The method of claim 12, wherein improving the health of the animal comprises reducing mortality in the animal as compared to an animal not administered the strain.

14. The method of claim 9, wherein the administering prevents necrotic enteritis in the animal.

15. The method of claim 9, wherein the administering reduces or ameliorates symptoms associated with a pathogenic infection in the animal.

16. The method of claim 9, wherein the administering decreases antibiotic use in an animal production system.

17. The method of claim 9, wherein the Bacillus velezensis PTA-127643 is present as spores.

18. The method of claim 9, wherein the animal is selected from the group consisting of bovine, equine, poultry, fish, and shellfish.

19. The method of claim 9, wherein the composition further comprises at least one of an animal feed ingredient, a supplement, a milk replacer, an enzyme, a prebiotic, and/or a probiotic.

20. The method of claim 9, wherein the composition is fed to the animal, at a rate of about 1.0 g of Bacillus velezensis PTA-127643/head animal/day.
